(19) United States
(12) Patent Application Publication
(10) Pub. No.: US 2020/0330144 A1
(43) Pub. Date: Oct. 22, 2020

(54) CONTROL DEVICE, TREATMENT SYSTEM AND OPERATION METHOD OF CONTROL DEVICE

(71) Applicant: OLYMPUS CORPORATION, Tokyo (JP)

(72) Inventors: Shohei MORIWAKI, Akishima-shi (JP); Yoshitaka HONDA, Hachioji-shi (JP); Tsuyoshi HAYASHIDA, Hachioji-shi (JP); Toshifumi KATSURAGI, Hachioji-shi (JP); Kazue TANAKA, Sagamihara-shi (JP); Sadayoshi TAKAMI, Hachioji-shi (JP)

(73) Assignee: OLYMPUS CORPORATION, Tokyo (JP)

(21) Appl. No.: 16/919,598

(22) Filed: Jul. 2, 2020

Related U.S. Application Data
(63) Continuation of application No. PCT/JP2018/001162, filed on Jan. 17, 2018.

(51) Int. Cl. A61B 18/10; A61B 18/12

(52) U.S. Cl. CPC A61B 18/10; A61B 2017/00973; A61B 18/1206

(57) ABSTRACT

A generator includes a control device including a processor, and outputs electrical energy to a treatment instrument. The processor determines whether a treatment target has been heat-denatured prior to receiving an output command, and, based on determination, selects, as an operation state of the treatment instrument, one of a first mode if it is determined that the treatment target has not been heat-denatured and a second mode if it is determined that the treatment target has been heat-denatured. The processor operates the treatment instrument at the selected operation state by controlling the output of the electrical energy to the treatment instrument.

### CROSS-REFERENCE TO RELATED APPLICATIONS

This is a Continuation Application of PCT Application No. PCT/JP2018/001162, filed Jan. 17, 2018, the entire contents of which are incorporated herein by reference.

### BACKGROUND

### 2. Description of the Related Art

US 2012/0078139A1 discloses a treatment system including a treatment instrument configured to grasp a treatment target such as a living tissue between a pair of grasping pieces of an end effector, and a generator configured to supply electrical energy to the treatment instrument. In this treatment system, bipolar electrodes are provided in the end effector of the treatment instrument, and a high frequency current is applied as treatment energy from the end effector to the treatment target through the supply of electrical energy from the generator to the bipolar electrodes. As a result, the treatment target is heat-denatured and sealed by Joule heat or the like caused by the high-frequency current.

### SUMMARY

Exemplary embodiments relate to a control device that can be used together with a treatment instrument and a generator. The present invention also relates to a treatment system including the treatment instrument and the generator, and relates to an operation method of the control device. In a control device of a generator, the generator can be configured to output electrical energy to a treatment instrument, thereby applying treatment energy that heat-denatures a treatment target from the treatment instrument to the treatment target, the control device including a processor configured to cause the generator to output the electrical energy to the treatment instrument based on reception of an output command; after starting an output of the electrical energy to the treatment instrument, determine whether or not the treatment target has been heat-denatured prior to receiving the output command; based on determination of heat denaturation of the treatment target, select, as an operation state of the treatment instrument, one of a first mode in which the treatment target is sealed by the treatment energy if it is determined that the treatment target has not been heat-denatured and a second mode in which the treatment target is sealed by the treatment energy if it is determined that the treatment target has been heat-denatured; and operate the treatment instrument at the selected operation state by controlling the output of the electrical energy to the treatment instrument.

Exemplary embodiments can also include a treatment system including: a treatment instrument including an end effector configured to apply treatment energy that heat-denatures a treatment target; a generator configured to output electrical energy to the treatment instrument; and an endoscope system configured to acquire an image of the treatment target, the generator including a processor configured to: cause the generator to output the electrical energy to the treatment instrument based on reception of an output command; acquire from the endoscope system a parameter indicative of a reaction of the treatment target to an application of the treatment energy after starting an output of the electrical energy to the treatment instrument; determine, based on the acquired parameter, whether or not the treatment target has been heat-denatured prior to receiving the output command; and select an operation mode of the treatment instrument based on a result of determination of heat denaturation of the treatment target.

Exemplary embodiments also include an operation method of a control device of a generator, the generator being configured to output electrical energy to a treatment instrument, thereby applying treatment energy that heat-denatures a treatment target from the treatment instrument to the treatment target, the method including: outputting the electrical energy from the generator to the treatment instrument based on reception of an output command; after starting an output of the electrical energy to the treatment instrument, determining whether or not the treatment target has been heat-denatured prior to receiving the output command; based on determination of heat denaturation of the treatment target, selecting, as an operation state of the treatment instrument, one of a first mode in which the treatment target is sealed by the treatment energy if it is determined that the treatment target has not been heat-denatured and a second mode in which the treatment target is sealed by the treatment energy if it is determined that the treatment target has been heat-denatured; and operating the treatment instrument at a selected operation state by controlling the output of the electrical energy to the treatment instrument.

Advantages of the invention will be set forth in the description which follows, and in part will be obvious from the description, or may be learned by practice of the invention. The advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a schematic diagram showing a treatment system according to an exemplary embodiment.

FIG. 2 is a block diagram schematically showing a configuration of the supply of electrical energy to a treatment instrument according to an exemplary embodiment.

FIG. 3 is a flowchart showing processing performed by a processor according to an exemplary embodiment in an operation control of the treatment instrument.

FIG. 4 is a flowchart showing processing performed by the processor according to an exemplary embodiment in determining heat denaturation of a treatment target.

FIG. 5 is a schematic diagram showing an example of a variation with time in an impedance of an electrical path of first electrical energy in an exemplary embodiment.

FIG. 6 is a flowchart showing processing performed by the processor according to an exemplary embodiment in an output control on the electrical energy based on a determination result in FIG. 4.

FIG. 7 is a schematic diagram showing an example of a target trajectory of an output voltage to bipolar electrodes in an exemplary embodiment.

FIG. 8 is a schematic diagram showing a pattern of determination of heat denaturation of the treatment target in the processor according to an exemplary embodiment.

FIG. 9 is a schematic diagram showing an example of a variation with time in electric power output to bipolar electrodes in an exemplary embodiment.

FIG. 10 is a schematic diagram showing an example of a variation with time in an output current to bipolar electrodes in an exemplary embodiment.

FIG. 11 is a schematic diagram showing an example of a variation with time in a temperature of a heater in an exemplary embodiment.

FIG. 12 is a schematic diagram showing a configuration of one of grasping pieces according to an exemplary embodiment.

FIG. 13 is a schematic diagram showing an example of a variation with time in ON-OFF states of an output of electrical energy to the treatment instrument in an exemplary embodiment.

FIG. 14 is a schematic diagram showing an example of a variation with time in a pressing force from a tissue to an end effector in an exemplary embodiment.

FIG. 15 is a schematic diagram showing an example of a variation with time in a gap between grasping pieces in an exemplary embodiment.

FIG. 16 is a schematic diagram for explaining a setting of a determination parameter by a processor according to an exemplary embodiment.

FIG. 17 is a schematic diagram showing a pattern of an output control on electrical energy based on a result of determination of heat denaturation of a treatment target in the processor according an exemplary embodiment.

FIG. 18 is a schematic diagram showing an example of a target trajectory of an output voltage to bipolar electrodes in an exemplary embodiment.

FIG. 19 is a schematic diagram showing an example of a target trajectory of an output voltage to bipolar electrodes in an exemplary embodiment.

FIG. 20 is a schematic diagram showing an example of a target trajectory of an impedance of an electrical path of first electrical energy in an exemplary embodiment.

FIG. 21 is a schematic diagram showing an example of a variation with time in output electric power to bipolar electrodes in an exemplary embodiment.

FIG. 22 is a schematic diagram showing an example of a variation with time in output electric power to bipolar electrodes in an exemplary embodiment.

FIG. 23 is a schematic diagram showing an example of a variation with time in ON-OFF states of an output of first electrical energy in an exemplary embodiment.

FIG. 24 is a schematic diagram showing an example of a target trajectory of a temperature of a heater in an exemplary embodiment.

FIG. 25 is a schematic diagram showing an example of a variation with time in a temperature of a heater in an exemplary embodiment.

FIG. 26 is a schematic diagram showing an example of a target trajectory of a temperature of a heater in an exemplary embodiment.

FIG. 27 is a schematic diagram showing an example of a variation with time in ON-OFF states of an output of second electrical energy in an exemplary embodiment.

FIG. 28 is a schematic diagram showing a configuration for changing grasping force of an end effector in an exemplary embodiment.

FIG. 29 is a block diagram schematically showing a configuration for moving a stopper in the configuration of FIG. 28.

FIG. 30 is a schematic diagram showing a configuration of an end effector according to an exemplary embodiment.

FIG. 31 is a schematic diagram showing an example of a target trajectory of an output voltage to bipolar electrodes in an exemplary embodiment.

FIG. 32 is a schematic diagram showing an example of determination of heat denaturation of a treatment target by a processor according to an exemplary embodiment.

FIG. 33 is a schematic diagram showing an example of the setting values of a first mode, a second mode, and a third mode set by the processor according to an exemplary embodiment.

FIG. 34 is a flowchart showing processing performed by a processor according to an exemplary embodiment. in an operation control of the treatment instrument.

### DETAILED DESCRIPTION

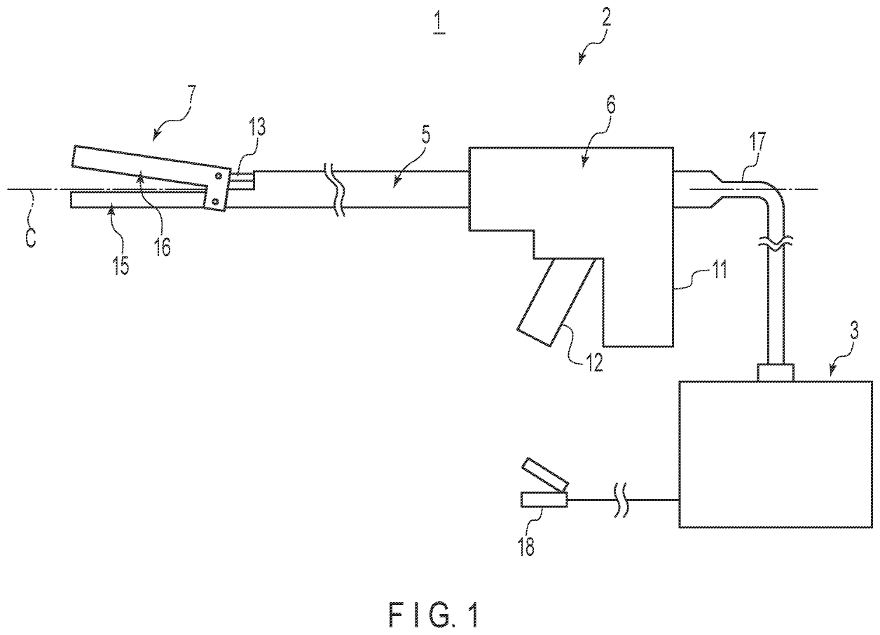

FIG. 1 is a diagram showing a treatment system 1 of the embodiment. As shown in FIG. 1, the treatment system 1 includes a treatment instrument 2 and an electric power source device (generator) 3 configured to output electrical energy to the treatment instrument 2. In this embodiment, when the treatment instrument 2 is used, the electric power source device 3 is also used in combination. The treatment instrument 2 includes a shaft 5, the shaft 5 having a longitudinal axis C as a central axis. A holdable housing 6 is connected to one end side (proximal side) of the shaft 5 in a direction along the longitudinal axis C. Further, an end effector 7 is connected to the other end of the shaft 5 opposite to the end where the housing 6 is located, i.e., connected to a distal end of the shaft 5. A grip 11 is provided in the housing 6, and a handle 12 is rotatably attached to the housing 6. The handle 12 rotates relative to the housing 6 so that the handle 12 opens or closes relative to the grip 11.

The end effector 7 includes a pair of grasping pieces 15 and 16. In the treatment instrument 2, a movable member 13 extends along the longitudinal axis C through an inside or outside of the shaft 5. One end (distal end) of the movable member 13 is connected to the end effector 7, and the other end (proximal end) of the movable member 13 is connected to the handle 12 inside the housing 6. By opening or closing the handle 12 with respect to the grip 11, the movable member 13 moves along the longitudinal axis C of the shaft 5, so that the pair of grasping pieces 15 and 16 open or close. Thus, a living tissue such as a blood vessel can be grasped as a treatment target between the grasping pieces 15 and 16. One of the grasping pieces 15 and 16 is rotatably attached to the distal portion of the shaft 5. The other of the grasping pieces 15 and 16 may be integral with or fixed to the shaft 5, or may be rotatably attached to the distal portion of the shaft 5. In one example, a rod member (not shown) extends inside the shaft 5, and a protruding portion of the rod member distally protruding from the distal end of the shaft 5 forms the other of the grasping pieces 15 and 16.

In one example, an operation member (not shown), such as a rotary knob, is attached to the housing 6. By rotating the operation member with respect to the housing 6, the shaft 5 and the end effector 7 are rotated with respect to the housing 6 about the longitudinal axis C. In another example, an operation member (not shown), such as a dial, is provided in the housing 6, and the end effector 7 is bent or curved with respect to the shaft 5 and the longitudinal axis C in accordance with an operation using the operation member. In this case, a relay member (not shown) provided in the end effector 7 is attached to the shaft 5 in a bendable or curvable manner. One of the grasping pieces 15 and 16 is rotatably attached to the relay member. The other of the grasping pieces 15 and 16 may be integrally fixed to the relay member or may be rotatably attached to the relay member. Further, a rod member (not shown) may extend inside the relay member, and the protruding portion of the rod member distally protruding from the distal end of the relay member may form the other of the grasping pieces 15 and 16.

One end of a cable 17 is connected to the housing 6. The other end of the cable 17 is separably connected to the electric power source device (generator) 3. The treatment system 1 is also provided with a footswitch 18 as an operation member separate from the treatment instrument 2. The footswitch 18 is electrically connected to the electric power source device 3. An operation for outputting electrical energy from the electric power source device 3 to the treatment instrument 2 is input via the footswitch 18. In one example, instead of the footswitch 18 or in addition to the footswitch 18, an operation button or the like is attached to the housing 6 as an operation member. In the operation member provided in the treatment instrument 2, an operation to output electrical energy from the electric power source device 3 to the treatment instrument 2 is input.

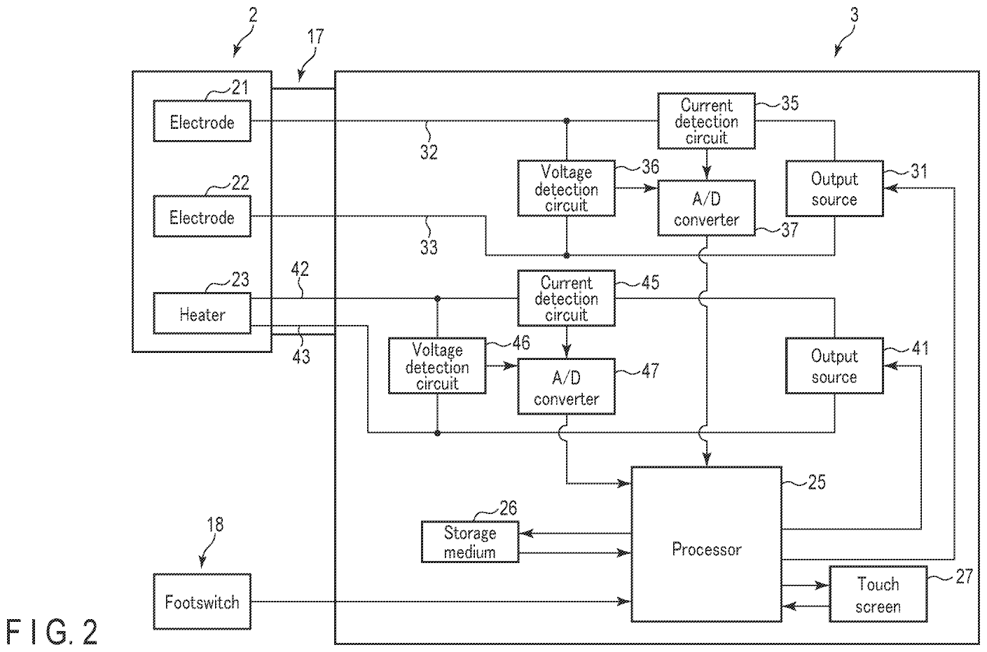

FIG. 2 is a diagram showing a configuration of the supply of electrical energy to the treatment instrument 2. As shown in FIG. 2, in the treatment instrument 2 of this embodiment, an electrode 21 is provided in the grasping piece 15, and an electrode 22 is provided in the grasping piece 16. The electrodes 21 and 22 are bipolar electrodes provided in the end effector 7. In the end effector 7, at least one of the grasping pieces 15 or 16 is provided with a heater 23 as a heating body.

The electric power source device 3 includes a processor (controller) 25 and a storage medium 26. The processor 25 is formed of an integrated circuit including a CPU (Central Processing Unit), an ASIC (Application Specific Integrated Circuit), an FPGA (Field Programmable Gate Array), or the like. Only one processor 25 may be provided in the electric power source device 3, or, alternatively, a plurality of processors 25 may be provided in the electric power source device 3. In this embodiment, the processor 25 constitutes a control device that controls the treatment system 1. The processing in the processor 25 is performed in accordance with a program stored in the processor 25 or the storage medium 26. The storage medium 26 stores a processing program for use in the processor 25, and parameters, functions, tables, and the like for use in calculations by the processor 25. The processor 25 receives an output command, i.e., a command signal, when an operation is input through the operation member such as the footswitch 18. Based on the reception of the output command, the processor 25 determines that an operation has been input via the operation member (18), that is, the operation input has been switched from OFF to ON, and causes electrical energy to be output from the electric power source device 3 to the treatment instrument 2.

The electric power source device 3 includes an output source (high-frequency electric power source) 31. The output source (high-frequency electric power source) 31 includes a waveform generator, a conversion circuit, a transformer, and the like, and forms a drive circuit (high-frequency drive circuit). The output source 31 is configured to convert electric power from a battery electric power source, a commercial electric power source, or the like into high-frequency electric power (high-frequency electrical energy), which is first electrical energy, and to output the first electrical energy. The output source 31 is electrically connected to the electrode 21 of the grasping piece 15 via an electrical path 32 and is electrically connected to the electrode 22 of the grasping piece 16 through an electrical path 33. Each of the electrical paths 32 and 33 extends inside the cable 17, inside the housing 6, and inside the shaft 5.

The first electrical energy output from the output source 31 is supplied to the electrodes (bipolar electrodes) 21 and 22 via the electrical paths 32 and 33. When the first electrical energy is output from the output source 31 in a state in which the treatment target is grasped between the grasping pieces 15 and 16, a high-frequency current flows through the treatment target between the electrodes 21 and 22. At this time, the electrodes 21 and 22 have electric potentials different from each other. The treatment target is heat-denatured and sealed by the Joule heat caused by the high-frequency current. Therefore, through the supply of the first electrical energy to the electrodes 21 and 22, a high-frequency current is applied as treatment energy from the end effector 7 to the treatment target to heat-denature the treatment target. In a state in which a high-frequency current is applied to the treatment target, the processor controls the output from the output source 31 and controls the supply of the first electrical energy to the electrodes 21 and 22.

The electric power source device 3 also includes a current detection circuit 35, a voltage detection circuit 36, and an A/D converter 37. The current detection circuit 35 detects an output current I_HF from the output source 31 to the electrodes 21 and 22, and the voltage detection circuit 36 detects an output voltage V_HF to the electrodes and 22. The A/D converter 37 converts analog signals indicative of a current value of the output current I_HF, detected by the current detection circuit 35, and analog signals indicative of a voltage value of the output voltage V_HF, detected by the voltage detection circuit 36, into digital signals, and transmits the converted digital signals to the processor 25. As a result, the processor 25 acquires information on the output current I_HF and the output voltage V_HF from the output source 31.

The processor 25 also calculates an impedance Z_HF of the circuit through which the high-frequency current (output current I_HF) flows, i.e., the impedance Z_HF of the electrical path of the first electrical energy, based on the output current I_HF and the output voltage V_HF from the output source 31. The impedance Z_HF varies in accordance with the impedance between the electrodes 21 and 22. In a state in which the treatment target is grasped between the grasping pieces 15 and 16, the impedance Z_HF varies in accordance with the impedance of the treatment target. In one example, the processor 25 calculates output electric power P_HF from the output source 31 based on the output current I_HF and the output voltage V_HF from the output source 31. In one example, the processor 25 calculates a phase difference Δφ between the output current I_HF and output voltage V_HF based on the output current I_HF and the output voltage V_HF. The processor 25 performs feedback control on the output of the first electrical energy from the output source 31 to the electrodes 21 and 22 based on any of the parameters associated with the first electrical energy, such as the output current I_HF, the output voltage V_HF, the output electric power P_HF, the impedance Z_HF, and the phase difference Δφ.

The electric power source device 3 also includes an output source (heater output source) 41. The output source (heater electric power source) 41 includes a conversion circuit, a relay circuit, a transformer, and the like, and forms a drive circuit (heater drive circuit). The output source 41 is configured to convert electric power from a battery electric power source, a commercial electric power source, or the like into DC electric power or AC electric power, which is second electrical energy different from the first electrical energy, and to output the second electrical energy. The output source 41 is electrically connected to the heater 23 via electrical paths 42 and 43. Each of the electrical paths 42 and 43 extends inside the cable 17, inside the housing 6, and inside the shaft 5.

The second electrical energy output from the output source 41 is supplied to the heater 23 via the electrical paths 42 and 43. As a result, heat is generated in the heater 23. When the second electrical energy is output from the output source 41 in a state in which the treatment target is grasped between the grasping pieces 15 and 16, the heat generated by the heater 23 is applied to the treatment target. Then, the treatment target is heat-denatured and sealed by the heat from the heater 23. Therefore, through the supply of the second electrical energy to the heater 23, the heat from the heater 23 is applied to the treatment target from the end effector 7 as treatment energy to heat-denature the treatment target. In a state in which the heat from the heater 23 is applied to the treatment target, the processor 25 controls the output from the output source 41 and controls the supply of the second electrical energy to the heater 23. The electric power source device 3 also includes a current detection circuit 45, a voltage detection circuit 46, and an A/D converter 47. The current detection circuit 45 detects an output current I_HT from the output source 41 to the heater 23, and the voltage detection circuit 46 detects an output voltage V_HT to the heater. The A/D converter 47 converts analog signals indicative of the current value of the output current I_HT detected by the current detection circuit 45 and analog signals indicative of the voltage value of the output voltage V_HT detected by the voltage detection circuit 46 into digital signals, and transmits the converted digital signals to the processor 25. As a result, the processor 25 acquires information on the output current I_HT and the output voltage V_HT from the output source 41.

The processor 25 also calculates an impedance Z_HT of the electrical path of the second electrical energy, based on the output current I_HT and the output voltage V_HT from the output source 41. The processor 25 calculates a resistance R_HT of the heater 23 based on the impedance Z_HT. The resistance R_HT of the heater 23 varies in accordance with a temperature T_HT of the heater 23. A function or a table indicative of the relationship between the temperature T_HT and the resistance R_HT of the heater is stored in the storage medium 26 or the like. The processor 25 calculates the temperature T_HT of the heater 23 based on the calculated resistance R_HT and the stored relationship between the temperature T_HT and the resistance R_HT. In one example, the processor 25 calculates output electric power P_HT from the output source 41 based on the output current I_HT and the output voltage V_HT from the output source 41. The processor 25 performs feedback control on the output of the second electrical energy from the output source 41 to the heater based on any of the parameters associated with the second electrical energy, such as the output current I_HT, the output voltage V_HT, the output electric power P_HT, and the temperature T_HT (the resistance R_HT).

In the present embodiment, the electric power source device 3 is provided with a touch screen 27. The touch screen 27 functions as an input unit through which settings related to the output from each of the output sources 31 and 41, such as the output level, are input. The touch screen 27 also functions as a display unit to display information related to the output from each of the output sources 31 and 41, such as the output current I_HF and the output voltage V_HF from the output source 31, and the output current I_HT and the output voltage V_HT from the output source 41.

Next, the operation and effect of the control device to control the treatment system 1 will be described. When performing treatment using the treatment system 1, the treatment instrument 2 is connected to the electric power source device 3 via the cable 17. The operator holds the housing 6 and inserts the end effector 7 into an abdominal cavity, a pleural cavity, and the like. Then, the handle 12 is closed with respect to the grip 11 in a state in which a treatment target such as a living tissue is located between the grasping pieces 15 and 16. As a result, the space between the grasping pieces 15 and 16 is closed, and a treatment target such as a blood vessel is grasped between the grasping pieces 15 and 16. When an operation is input by the operation member, such as the footswitch 18, in a state in which the treatment target is grasped, the processor 25 controls the HF (high-frequency) output, which is the output of the first electrical energy from the output source 31 to the electrodes 21 and 22, and the HT (heater) output, which is the output of the second electrical energy from the output source 41 to the heater 23, as will be described later. When the first electrical energy is supplied to the electrodes 21 and 22, a high-frequency current flows to the treatment target as described above, and when the second electrical energy is supplied to the heater 23, the heat generated by the heater 23 is applied to the treatment target as described above. When at least one of the high-frequency current or the heat of the heater 23 is applied to the treatment target as treatment energy, the treatment target is heat-denatured and sealed.

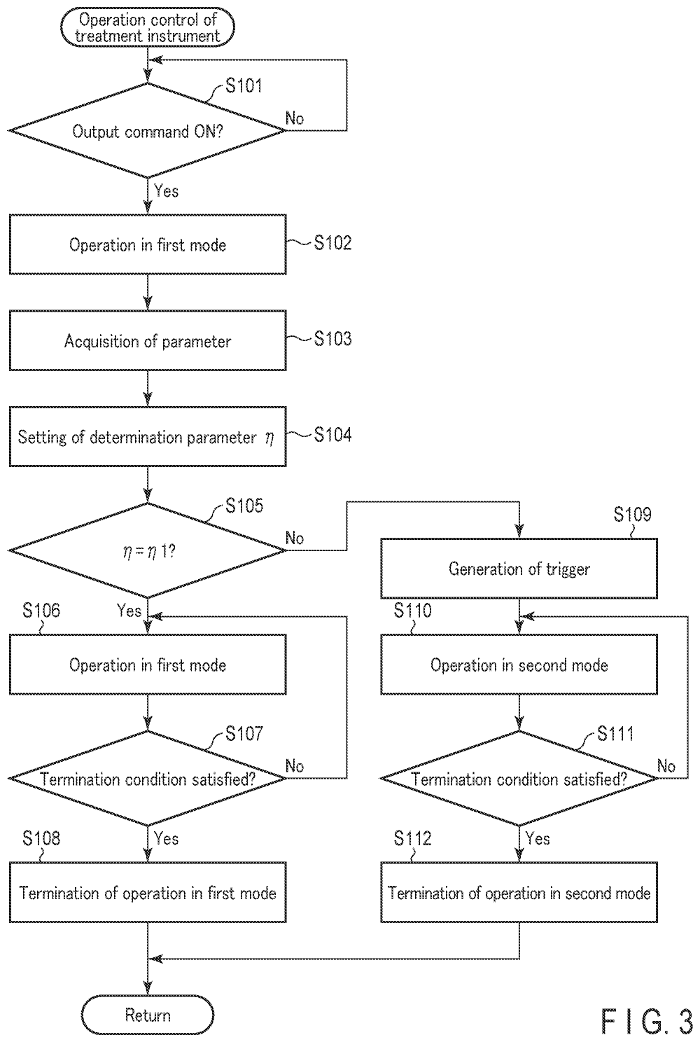

FIG. 3 is a flowchart showing the processing performed by the processor 25 in an operation control of the treatment instrument 2. As shown in FIG. 3, the processor determines whether an operation is input by the operation member such as the footswitch 18, and whether an output command is generated. That is, the processor 25 determines whether the output command is ON or OFF (S101). If no operation is input and no output command is generated (S101—No), the processing returns to S101. That is, the processor 25 stands by until an output command is generated. When an output command is generated by an operation input through the operation member (S101—Yes), the processor 25 receives the output command. The processor 25 then starts the output of the electrical energy from the electric power source device 3 to the treatment instrument 2 and causes the treatment instrument 2 to operate in a first mode (S102). When the treatment instrument 2 is operated in the first mode, the treatment energy, such as the high-frequency current and the heat of the heater 23, is applied to the treatment target, and the treatment target is heat-denatured by the treatment energy.

When the treatment instrument 2 is operated in the first mode, the processor 25 acquires a parameter indicative of a reaction of the treatment target to the application of the treatment energy (S103). The processor 25 sets a determination parameter η based on the acquired parameter (S104). The determination parameter η is a parameter indicative of whether or not the treatment target has been heat-denatured by the previous application of the treatment energy or the like prior to receiving the output command, that is, before S101. The parameter is set to a value η1 or a value η2. In the present embodiment, if it is determined that the treatment energy has not been applied to the treatment target and the treatment target has not been heat-denatured before the output command is received, the processor 25 sets the determination parameter η to the value η1. On the other hand, if it is determined that the treatment energy has been applied to the treatment target one or more times, and the treatment target has been heat-denatured before the output command is received, the processor 25 sets the determination parameter η to the value η2. When the determination parameter η is set, the processing proceeds to S105.

If the determination parameter η is set to the value η1 (S105—Yes), the processor 25 maintains the operation of the treatment instrument 2 in the first mode (S106). That is, if the processor 25 determines that the treatment target has not been heat-denatured, it selects the first mode as the operation state of the treatment instrument 2. The processor 25 then causes the treatment instrument 2 to operate in the selected first mode to seal the treatment target with the treatment energy. Then, the processor 25 determines whether or not the termination condition to terminate the first mode is satisfied (S107). If the termination condition is not satisfied (S107—No), the processing returns to S106. Thus, until the termination condition is satisfied, the processor 25 continuously causes the treatment instrument 2 to operate in the first mode. On the other hand, if the termination condition is satisfied (S107—Yes), the processor 25 terminates the operation in the first mode of the treatment instrument 2 (S108).

If the determination parameter η is set to the value η2 (S105—No), the processor 25 generates a trigger (S109). Based on the generation of the trigger, the processor 25 switches the operation state of the treatment instrument 2 from the first mode to the second mode (S110). At this time, the processor 25 switches the operation state of the treatment instrument 2 to the second mode by switching the output state of the electrical energy from the electric power source device (generator) 3. Accordingly, if the processor 25 determines that the treatment target has been heat-denatured, the processor 5 selects the second mode as the operation state of the treatment instrument 2. The processor 25 then causes the treatment instrument 2 to operate in the selected second mode to seal the treatment target with the treatment energy. Then, the processor 25 determines whether or not the termination condition to terminate the second mode is satisfied (S111). If the termination condition is not satisfied (S111—No), the processing returns to S110. Thus, until the termination condition is satisfied, the processor 25 continuously causes the treatment instrument 2 to operate in the second mode. On the other hand, if the termination condition is satisfied (S111—Yes), the processor 25 terminates the operation of the treatment instrument 2 in the second mode (S112).

In one example, upon termination of the operation of the treatment instrument 2 in each of the first mode and the second mode, the processor 25 ceases the output of the electrical energy to the treatment instrument 2. In another example, upon termination of the operation of the treatment instrument 2 in each of the first and second modes, the processor 25 switches the output state of the electrical energy to the treatment instrument 2, and causes the treatment instrument 2 to operate in a mode different from both the first mode and the second mode in which the treatment target is sealed. At this time, the processor 25 causes the treatment instrument 2 to operate, for example, in a mode in which the treatment target is incised.

[First Example for Realizing Operation Control]

A first example of realizing the operation control of the treatment instrument 2 shown in FIG. 3 will be described. In this example, the processor 25 controls the output of electrical energy to the treatment instrument 2 in the following manner, thereby controlling the operation of the treatment instrument 2 described above. Further, in the present example, in the treatment for sealing a treatment target, the processor 25 sequentially performs an output control in a first phase, an output control in a second phase, and an output control in a third phase according to the output of the electrical energy. The processor 25 performs the output control in the first phase until a predetermined time tref elapses from the start of the output of the electrical energy. The predetermined time tref is a short time, for example, about 100 ms.

When the output of the electrical energy is started, and the application of the treatment energy, such as the high-frequency current and the heat of the heater 23, to the treatment target is started, the impedance of the treatment target decreases with time until the moisture of the treatment target has evaporated. Therefore, the impedance Z_HF of the electrical path of the first electrical energy decreases with time. When the moisture of the treatment target begins to evaporate, the impedance of the treatment target begins to increase with time, so that the impedance Z_HF begins to increase with time. For this reason, a minimum value Zmin_HF of the impedance Z_HF, at which the impedance Z_HF changes from a state in which the impedance decreases with time to a state in which the impedance increases with time, is generated at or immediately before or after the start of evaporation of the moisture of the treatment target. The processor 25 performs the output control in the second phase until the minimum value Zmin_HF of the impedance Z_HF is detected, after the termination of the output control in the first phase. The processor 25 detects a time of switching from the state in which the impedance Z_HF decreases with time to the state in which the impedance Z_HF increases with time, and determines that the impedance Z_HF becomes the minimum value Zmin_HF at the time of the switching based on the fact that the impedance Z_HF has increased by a reference value or more since the time of switching. Therefore, the minimum value Zmin_HF is detected when the impedance Z_HF is increased from the minimum value Zmin_HF by some extent. After detecting the minimum value Zmin_HF, the processor 25 performs the output control in the third phase until the termination condition is satisfied.

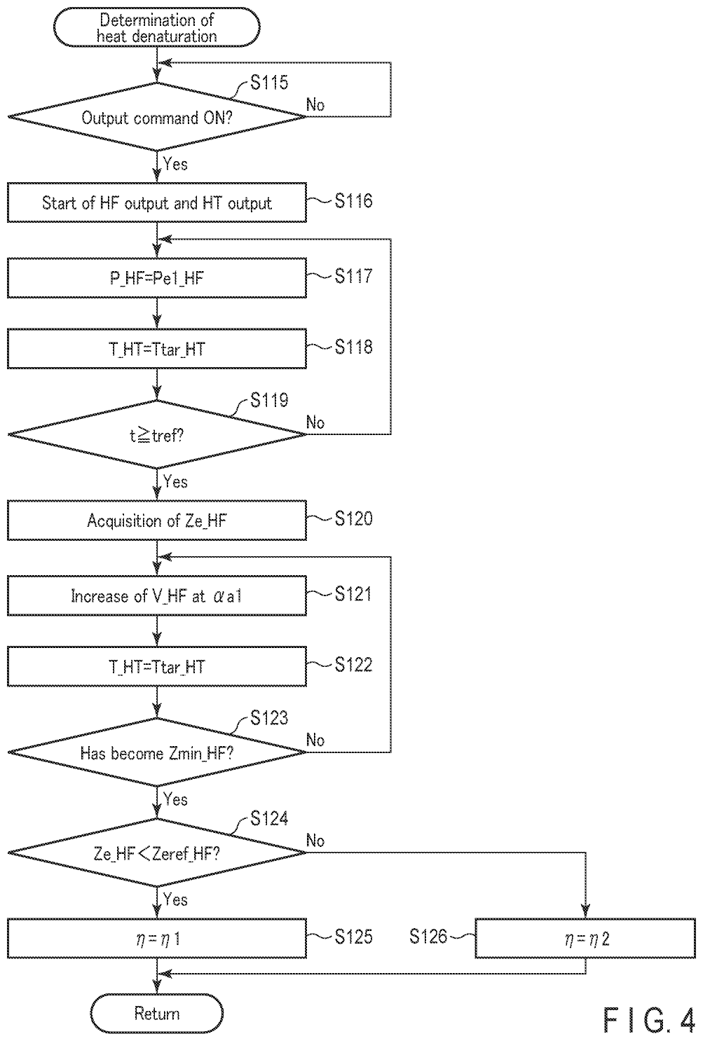

FIG. 4 is a flowchart showing processing performed by the processor 25 in determining heat denaturation of a treatment target. The processing in FIG. 4 of this example corresponds to the processing of S101 to S104 in FIG. 3. In this example, the processor 25 determines the heat denaturation of the treatment target before the output control in the third phase is started, that is, while the output control in the first phase and the output control in the second phase are performed. As shown in FIG. 4, in this example also, when an output command is generated by an operation input through the operation member (S115—Yes), the processor 25 starts the output of the electrical energy to the treatment instrument 2, and starts the output control in the first phase. In this example, the processor starts an output (HF output) of the first electrical energy to the electrodes 21 and 22 and an output (HT output) of the second electrical energy to the heater 23 based on the output command (S116). As a result, the treatment instrument 2 is operated in the first mode.

In this example, the processor 25 sets an initial value (setting value) Pe_HF for the output electric power P_HF to the electrodes 21 and 22 in the first phase. At this time, the processor 25 sets the initial value Pe_HF to a constant value Pe1_HF. Then, the processor 25 performs an output control on the first electrical energy to maintain the output electric power P_HF at the set initial value Pe1_HF over time (S117). The processor 25 also sets a target temperature (setting value) Ttar_HT for the temperature T_HT of the heater 23. Then, the processor 25 performs an output control on the second electrical energy so that the temperature T_HT of the heater 23 reaches the set target temperature Ttar_HT and is maintained at the target temperature Ttar_HT (S118). At this time, the processor 25 performs PD control or PID control at the target temperature Ttar_HT for the output of the second electrical energy. Then, the processor 25 determines whether or not the time t from the start of the output of the electrical energy is equal to or greater than the predetermined time tref (S119). That is, the processor 25 determines whether or not a predetermined time tref has elapsed since the output start time (t=0).

If the predetermined time tref has not elapsed since the output start time (S119—No), the processing returns to S117, and the processor 25 sequentially performs the process of S117 and the subsequent processes. Therefore, from the output start time of the electrical energy until the predetermined time tref has elapsed, the processor 25 continuously performs the output control on the first electrical energy to bring the output electric power P_HF to the initial value Pe1_HF and the output control on the second electrical energy to bring the temperature T_HT to the target temperature Ttar_HT. If the predetermined time tref has elapsed since the output start time (S119—Yes), the processor 25 acquires an initial value Ze_HF of the impedance Z_HF as a value indicative of the impedance Z_HF at or immediately after the output start time (S120). In this example, the processor 25 acquires the initial value Ze_HF as a parameter indicative of a reaction of the treatment target to the application of the treatment energy. The initial value Ze_HF of the impedance Z_HF may be the impedance Z_HF at any point of time in the first phase, or may be an average value or an intermediate value of the impedance Z_HF in the first phase. The impedance Z_HF including the initial value Ze_HF is a parameter for use in a feedback control of the output of the first electrical energy. If the predetermined time tref has elapsed since the output start time (S119—Yes), the processor 25 switches the output control in the first phase to the output control in the second phase.

In the second phase, the processor 25 sets a rate aa of increase (setting value) with time in the output voltage V_HF to the electrodes 21 and 22, and sets a target trajectory of the output voltage V_HF, along which the output voltage V_HF increases with time at the rate aa of increase. In this example, the processor 25 sets the rate aa of increase to a value αa1. Then, according to the first electrical energy, the processor 25 performs an output control to increase the output voltage V_HF with time at the rate αa1 of increase (S121). In other words, the processor 25 performs the output control on the first electrical energy, so that the output voltage V_HF is brought along the target trajectory. Also, in the second phase, the processor 25 performs an output control on the second electrical energy so that the temperature T_HT of the heater 23 reaches the set target temperature Ttar_HT and is maintained at the target temperature Ttar_HT (S122). Then, the processor 25 determines whether or not the impedance Z_HF has become the minimum value Zmin_HF (S123). At this time, as described above, the processor 25 detects a time of switching from the state in which the impedance Z_HF decreases with time to the state in which the impedance Z_HF increases with time, and determines whether or not the impedance Z_HF has increased by a reference value or more since the time of switching.

If it is determined that the impedance Z_HF has not become the minimum value Zmin_HF (S123—No), the processing returns to S121, and the processor 25 sequentially performs the process of S121 and the subsequent steps. Thus, from the termination of the output control in the first phase (t=tref) until the minimum value Z_min is detected, the processor 25 continuously performs the output control on the first electrical energy to increase the output voltage V_HF with time at the rate αa1 of increase and the output control on the second electrical energy to bring the temperature T_HT to the target temperature Ttar_HT. If it is determined that the impedance Z_HF has become the minimum value Zmin_HF (S123—Yes), the processor 25 switches the output control in the second phase to the output control in the third phase. At this time, the processor 25 determines, by the commencement of the output control in the third phase, whether or not the treatment target has been heat-denatured by the previous application of the treatment energy or the like prior to receiving the output command. Then, the processor 25 sets the determination parameter η before the start of the output control in the third phase.

In the present example, the processor 25 determines the heat denaturation of the treatment target based on the initial value Ze_HF of the impedance Z_HF acquired in S120. In the determination of the heat denaturation of the treatment target, the processor 25 determines whether or not the initial value Ze_HF acquired as a parameter is smaller than the reference value Zeref_HF (S124). If the initial value Ze_HF is smaller than the reference value Zeref_HF (S124—Yes), the processor 25 sets the determination parameter η to the value η1, and determines that the treatment target has not been heat-denatured prior to receiving the output command (S125). On the other hand, if the initial value Ze_HF is equal to or greater than the reference value Zeref_HF (S124—No), the processor 25 sets the determination parameter η to the value η2, and determines that the treatment target has been heat-denatured prior to receiving the output command (S126).

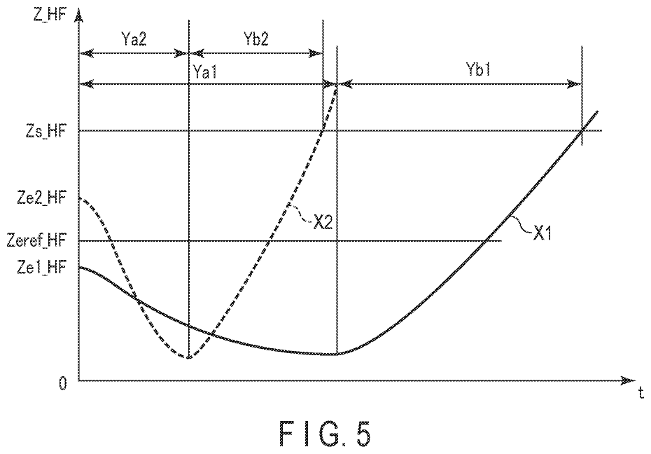

FIG. 5 shows an example of a variation with time in the impedance Z_HF in this example. In FIG. 5, the abscissa axis indicates time t with reference to the output start time of electrical energy, and the ordinate axis indicates the impedance Z_HF. FIG. 5 shows variations with time in the impedance Z_HF in the states X1 and X2, in which the states of the treatment target are different from each other. In the state X1, before the processor 25 receives an output command, the treatment energy has not been applied to the treatment target and the treatment target has not been heat-denatured. Therefore, in the state X1, the processor 25 applies treatment energy for the first time (at the initial time) to the treatment target based on the reception of the output command. On the other hand, in the state X2, before the processor 25 receives an output command, the treatment energy has been applied one or more times to the treatment target, and the treatment target has been heat-denatured by the application of the treatment energy. Therefore, in the state X2, the processor 25 applies treatment energy second and subsequent times to the heat-denatured treatment target based on the reception of the output command. In FIG. 5, the variation with time in the impedance Z_HF in the state X1 is indicated by the solid line, and the variation with time in the impedance Z_HF in the state X2 is indicated by the broken line.

In the state X2 in which the treatment target has been heat-denatured prior to the output command, the degree of moisture of the treatment target is less and the initial value Ze_HF of the impedance Z_HF is higher than in the state X1 in which the treatment target has not been heat-denatured prior to the output command. For example, in the example of FIG. 5, an initial value Ze2_HF in the state X2 is higher than an initial value Ze1_HF in the state X1. The initial value Ze1_HF in the state X1 is lower than the reference value Zeref_HF, and the initial value Ze2_HF in the state X2 is equal to or greater than the reference value Zeref_HF. Therefore, in accordance with the determination of S124 based on the initial value Ze_HF, the processor 25 sets the determination parameter η to the value η1 in the state X1, and determines that the treatment target has not been heat-denatured prior to receiving the output command. On the other hand, in the state X2, the processor 25 sets the determination parameter η to the value η2, and determines that the treatment target has been heat-denatured prior to receiving the output command. Thus, the processor 25 appropriately determines whether or not the treatment target has been heat-denatured prior to receiving the output command, and appropriately sets the determination parameter η.

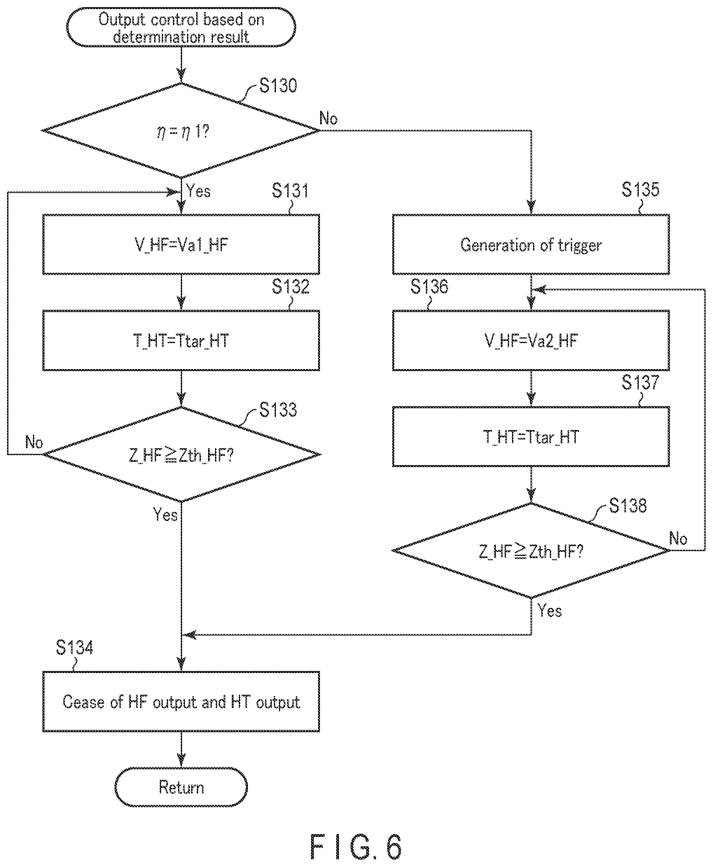

FIG. 6 is a flowchart showing processing performed by the processor 25 in the output control on the electrical energy based on the determination result in the processing shown in FIG. 4. The processing in FIG. 6 of this example corresponds to the processing of S105 to S112 in FIG. 3. In this example, the processor 25 sets the determination parameter η before the output control in the third phase is started. In the third phase, the processor 25 controls the output of the electrical energy based on the set determination parameter η. As shown in FIG. 6, in the third phase, the processor 25 determines whether the determination parameter η is set to the value η1 or η2 (S130).

If the determination parameter η is set to the value η1 (S130—Yes), that is, if it is determined that the treatment target has not been heat-denatured prior to receiving the output command, the processor 25 continues the operation of the treatment instrument 2 in the first mode. In this case, the processor 25 sets the target value (setting value) Va1_HF for the output voltage V_HF. Then, the processor 25 performs a constant voltage control on the output of the first electrical energy to maintain the output voltage V_HF over time at the target value Va1_HF (S131). Also in the third phase, the processor 25 performs the output control on the second electrical energy to bring the temperature T_HT to the target temperature Ttar_HT (S132). Then, the processor 25 determines whether or not the impedance Z_HF is equal to or greater than a threshold value Zth_HF (S133). That is, the processor 25 determines whether or not the impedance Z_HF has increased to the threshold value Zth_HF. The threshold value Zth_HF is set as a setting value associated with the termination condition, and the setting value is higher than the initial value Ze_HF.

If the impedance Z_HF is smaller than the threshold value Zth_HF (S133—No), the processing returns to S131, and the processor 25 sequentially performs the processing of S131 and the subsequent steps. Therefore, if the determination parameter η is set to the value η1, in the period from the termination of the output control in the second phase until the impedance Z_HF reaches the threshold value Zth_HF, the processor 25 continuously performs the constant voltage control on the first electrical energy to bring the output voltage V_HF to the target value Va1_HF and the output control on the second electrical energy to bring the temperature T_HT to the target temperature Ttar_HT. If the impedance Z_HF is equal to or greater than the threshold value Zth_HF (S133—Yes), the processor 25 ceases the output (HF output) of the first electrical energy to the electrodes 21 and 22 and the output (HT output) of the second electrical energy to the heater 23 (S134). As a result, the output control in the third phase terminates, and the operation of the treatment instrument 2 in the first mode terminates.

On the other hand, if the determination parameter η is set to the value η2 (S130—No), that is, if it is determined that the treatment target has been heat-denatured prior to receiving the output command, the processor 25 switches the operation state of the treatment instrument 2 from the first mode to the second mode. In this case, the processor 25 generates a trigger (S135). Then, the processor 25 sets a target value Va2_HF smaller than the target value Va1_HF for the output voltage V_HF. Then, the processor 25 performs a constant voltage control on the output of the first electrical energy to maintain the output voltage V_HF over time at the target value Va2_HF (S136). Also, if the determination parameter η is set to the value η2, in the third phase, the processor 25 performs the output control on the second electrical energy to bring the temperature T_HT to the target temperature Ttar_HT (S137). Then, as in the case where the determination parameter η is set to the value η1, the processor 25 determines whether or not the impedance Z_HF is equal to or greater than the threshold value Zth_HF (S138).

If the impedance Z_HF is smaller than the threshold value Zth_HF (S138—No), the processing returns to S136, and the processor 25 sequentially performs the processing of S136 and the subsequent steps. Therefore, if the determination parameter η is set to the value η2, from the termination of the output control in the second phase until the impedance Z_HF reaches the threshold value Zth_HF, the processor 25 continuously performs the constant voltage control on the first electrical energy to bring the output voltage V_HF to the target value Va2_HF and the output control on the second electrical energy to bring the temperature T_HT to the target temperature Ttar_HT. If the impedance Z_HF is equal to or greater than the threshold value Zth_HF (S138—Yes), the processor 25 ceases the output of the first electrical energy to the electrodes 21 and 22 and the output of the second electrical energy to the heater 23 (S134). As a result, the output control in the third phase terminates, and the operation of the treatment instrument 2 in the second mode terminates.

Since the output control on the first electrical energy is performed as described above, in this example, in the constant voltage control on the first electrical energy in the third phase, the processor 25 sets the target value (setting value) Va_HF of the output voltage V_HF to differing values for the first mode and the second mode. When the treatment instrument 2 is operating in the second mode, the processor 25 suppresses the output of the first electrical energy from the electric power source device (generator) 3 to the treatment instrument 2 as compared with the state in which the treatment instrument 2 is operating in the first mode. Thus, in the second mode, the application of the high-frequency current (treatment energy) to the treatment target is suppressed as compared with the first mode.

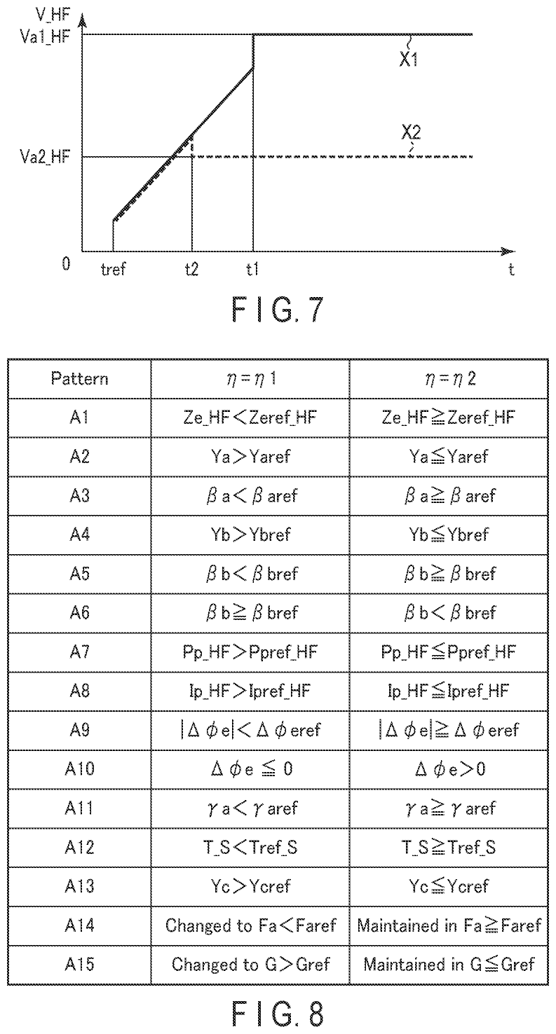

FIG. 7 shows an example of a target trajectory of the output voltage V_HF to the electrodes 21 and 22 in this example. In FIG. 7, the abscissa axis indicates time t with reference to the output start time, and the ordinate axis indicates the output voltage V_HF. FIG. 7 shows target trajectories of the output voltage V_HF in the second phase and the third phase, and the target trajectory of the output voltage V_HF in the state X1 is indicated by the solid line, while the target trajectory of the output voltage V_HF in the state X2 is indicated by the broken line. As shown in FIG. 7, the processor 25 of the present example performs the control to increase the output voltage V_HF with time at the rate αa1 of increase in the second phase in both the states X1 and X2. However, in the state X1 in which the determination parameter η is set to the value η1, the processor 25 performs a constant voltage control to maintain the output voltage V_HF at the target value Va1_HF in the third phase, and causes the treatment instrument 2 to operate in the first mode. On the other hand, in the state X2 in which the determination parameter η is set to the value η2, the processor 25 performs a constant voltage control to maintain the output voltage V_HF at the target value Va2_HF, which is smaller than the target value Va1_HF, in the third phase, and causes the treatment instrument 2 to operate in the second mode. Therefore, in the third phase, the output of the first electrical energy from the output source 31 to the electrodes 21 and 22 is suppressed in the state X2 as compared with the state X1. That is, in the state X2 in which the treatment target has been heat-denatured prior to the output command, the processor 25 suppresses the output of the first electrical energy to the treatment instrument as compared with the state X1 in which the treatment target has not been heat-denatured prior to the output command.

Furthermore, in the state X2 in which the treatment target has been heat-denatured prior to the output command, the time Ya elapsed from the output start time until the impedance Z_HF reaches the minimum value Zmin_HF is shorter than that in the state X1 in which the treatment target has not been heat-denatured prior to the output command (see FIG. 5). Therefore, as shown in FIG. 7, in the state X1, the impedance Z_HF increases from the minimum value Zmin_HF by a reference value or more at time t1, and the minimum value Zmin_HF is detected. The processor 25 switches the output control in the second phase to the output control in the third phase at the time t1. On the other hand, in the state X2, the impedance Z_HF increases from the minimum value Zmin_HF by a reference value or more at a time t2 prior to the time t1, and the minimum value Zmin_HF is detected. The processor 25 switches the output control in the second phase to the output control in the third phase at the time t2 before the time t1.

In the treatment for sealing the treatment target, treatment energy such as a high-frequency current may be applied to the same portion more than once as in the case of the state X2, depending on the size and type of the treatment target such as a blood vessel. In this case, in the second and subsequent applications of treatment energy, the treatment energy is again applied to the place where the treatment target has already been heat-denatured by the application of the treatment energy. In this example, in the state X1 in which the treatment energy is applied for the first time to the portion where the treatment energy has not been applied, the processor 25 determines that the treatment target has not been heat-denatured prior to the output command, and causes the treatment instrument to operate in the first mode. On the other hand, in the state X2 in which the treatment energy is again applied to the already heat-denatured portion, the processor 25 determines that the treatment target has been heat-denatured prior to the output command, and causes the treatment instrument to operate in the second mode. Then, in the second mode, the application of the high-frequency current (treatment energy) to the treatment target is suppressed as compared with the first mode via control of the output of the electrical energy by the processor 25. Therefore, in the state X2 in which the treatment energy is again applied to the already heat-denatured portion, the application of the high-frequency current to the treatment target is suppressed as compared with the state X1 in which the treatment energy is applied for the first time to a portion which has not been heat-denatured. Accordingly, in the state X2 in which the treatment energy is applied second and subsequent times to the already heat-denatured portion, the sealing performance and the like of the treatment target can be appropriately secured and the appropriate treatment performance can be exhibited.

[Pattern of Determination of Heat Denaturation of Treatment Target]

In the first example shown in FIGS. 4 to 7, the processor 25 acquires the initial value Ze_HF of the impedance Z_HF as a parameter indicative of a reaction of the treatment target to the application of the treatment energy, and determines the heat denaturation of the treatment target based on the initial value Ze_HF. However, in another example, the processor 25 may determine, based on a parameter other than the initial value Ze_HF, whether or not the treatment target has been heat-denatured prior to receiving the output command, and set the determination parameter η. FIG. 8 shows a pattern of determination by the processor 25 of heat denaturation of the treatment target. In the first example, as described above, the processor 25 makes a determination of the heat denaturation and sets the determination parameter η in accordance with a pattern A1.

In a second example, the processor 25 makes a determination of the heat denaturation in accordance with a pattern A2. In this case, based on a time Ya elapsed from the output start time until the impedance Z_HF becomes the minimum value Zmin_HF, the processor 25 determines the heat denaturation of the treatment target and sets the determination parameter η. In this example, if the time Ya is longer than a reference time Yaref, the processor 25 determines that the treatment target has not been heat-denatured prior to receiving the output command, and sets the determination parameter η to the value η1. On the other hand, if the time Ya is equal to or shorter than the reference time Yaref, the processor 25 determines that the treatment target has been heat-denatured prior to receiving the output command, and sets the determination parameter η to the value η2.

In the state X2 in which the treatment target has been heat-denatured prior to the output command, the time Ya which elapses from the output start time until the impedance Z_HF reaches the minimum value Zmin_HF is shorter than in the state X1 in which the treatment target has not been heat-denatured prior to the output command. For example, in the example of FIG. 5, the time Ya2 in the state X2 is shorter than the time Ya1 in the state X1. The time Ya1 in the state X1 is longer than the reference time Yaref, and the time Ya2 in the state X2 is equal to or shorter than the reference time Yaref. Therefore, in accordance with the determination based on the time Ya, in the state X1, the processor 25 determines that the treatment target has not been heat-denatured prior to the output command. On the other hand, in the state X2, the processor 25 determines that the treatment target has been heat-denatured prior to the output command. Thus, the processor 25 appropriately determines whether or not the treatment target has been heat-denatured prior to receiving the output command, and appropriately sets the determination parameter η.

In a third example, the processor 25 makes a determination of the heat denaturation in accordance with a pattern A3. In this case, based on a rate βa of decrease in the impedance Z_HF until the impedance reaches the minimum value Zmin_HF, the processor 25 determines the heat denaturation of the treatment target and sets the determination parameter η. In this example, if the impedance Z_HF gradually decreases to the minimum value Zmin_HF and the rate βa of decrease is smaller than a reference value βaref, the processor 25 determines that the treatment target has not been heat-denatured prior to receiving the output command, and sets the determination parameter η to the value η1. On the other hand, if the impedance Z_HF rapidly decreases to the minimum value Zmin_HF and the rate βa of decrease is equal to or greater than the reference value βaref, the processor 25 determines that the treatment target has been heat-denatured prior to receiving the output command, and sets the determination parameter η to the value η2.

In the state X2 in which the treatment target has been heat-denatured prior to the output command, the impedance Z_HF more rapidly decreases until the impedance reaches the minimum value Zmin_HF as compared with the state X1 in which the treatment target has not been heat-denatured prior to the output command. For example, in an example of FIG. 5, the rate βa2 of decrease in the state X2 is greater than the rate βa1 of decrease in the state X1. The rate βa1 of decrease in the state X1 is smaller than the reference value βaref, and the rate βa2 of decrease in the state X2 is equal to or greater than the reference value βaref. Therefore, in accordance with the determination based on the rate βa of decrease, in the state X1, the processor 25 determines that the treatment target has not been heat-denatured prior to the output command. On the other hand, in the state X2, the processor 25 determines that the treatment target has been heat-denatured prior to the output command. Thus, the processor 25 appropriately determines whether or not the treatment target has been heat-denatured prior to receiving the output command, and appropriately sets the determination parameter η.

In a fourth example, the processor 25 makes a determination of the heat denaturation in accordance with a pattern A4. In this case, based on the time Yb elapsed until the impedance Z_HF reaches the predetermined value Zs_HF from the minimum value Zmin_HF, the processor 25 determines the heat denaturation of the treatment target and sets the determination parameter η. In this example, if the time Yb is longer than a reference time Ybref, the processor 25 determines that the treatment target has not been heat-denatured prior to receiving the output command, and sets the determination parameter η to the value η1. On the other hand, if the time Yb is equal to or shorter than the reference time Ybref, the processor 25 determines that the treatment target has been heat-denatured prior to receiving the output command, and sets the determination parameter η to the value η2. The processor 25 sets the predetermined value Zs_HF to be higher than the initial value Ze_HF of the impedance Z_HF and lower than a threshold value Zth_HF of the termination condition.

In the state X2 in which the treatment target has been heat-denatured prior to the output command, the treatment target is already dried to some extent. Therefore, in the state X2 in which the treatment target has been heat-denatured prior to the output command, the impedance Z_HF may more rapidly increase from the minimum value Zmin_HF to the predetermined value Zs_HF as compared with the state X1 in which the treatment target has not been heat-denatured prior to the output command. In this case, for example, as shown in FIG. 5, the time Yb2 in the state X2 is shorter than the time Yb1 in the state X1. The time Yb1 in the state X1 is longer than the reference time Ybref, and the time Yb2 in the state X2 is equal to or shorter than the reference time Ybref. Therefore, in accordance with the determination based on the time Yb, the processor 25 appropriately determines whether the treatment target has been heat-denatured prior to receiving the output command, and appropriately sets the determination parameter η.

In a fifth example, the processor 25 makes a determination of the heat denaturation in accordance with a pattern A5. In this case, based on a rate βb of increase in the impedance Z_HF from the minimum value Zmin_HF, the processor 25 determines the heat denaturation of the treatment target and sets the determination parameter η. In this example, if the impedance Z_HF gradually increases from the minimum value Zmin_HF and the rate βb of increase is smaller than a reference value βbref, the processor 25 determines that the treatment target has not been heat-denatured prior to receiving the output command, and sets the determination parameter η to the value η1. On the other hand, if the impedance Z_HF rapidly increases from the minimum value Zmin_HF and the rate βb of increase is equal to or greater than the reference value βbref, the processor 25 determines that the treatment target has been heat-denatured prior to receiving the output command, and sets the determination parameter η to the value η2.

As described above, in the state X2 in which the treatment target has been heat-denatured prior to the output command, the impedance Z_HF may more rapidly increase from the minimum value Zmin_HF as compared with the state X1 in which the treatment target has not been heat-denatured prior to the output command. In this case, as in the example of FIG. 5, the rate βb2 of increase in the state X2 is greater than the rate βb1 of increase in the state X1. The rate βb1 of increase in the state X1 is smaller than the reference value βbref, and the rate βb2 of increase in the state X2 is equal to or greater than the reference value βbref. Thus, in accordance with the determination based on the rate βb of increase, the processor 25 appropriately determines whether or not the treatment target has been heat-denatured prior to receiving the output command, and appropriately sets the determination parameter η.

In a sixth example, the processor 25 makes a determination of the heat denaturation in accordance with a pattern A6. In this case, the processor 25 makes a determination opposite to the pattern A5 based on the rate βb of increase in the impedance Z_HF from the minimum value Zmin_HF. That is, in this example, if the rate βb of increase is equal to or greater than the reference value βbref, the processor 25 determines that the treatment target has not been heat-denatured prior to receiving the output command, and sets the determination parameter η to the value η1. On the other hand, if the rate βb of increase is smaller than the reference value βbref, the processor 25 determines that the treatment target has been heat-denatured prior to receiving the output command, and sets the determination parameter η to the value η2.

In the state X2 in which the treatment target has been heat-denatured prior to the output command, the impedance Z_HF is already high to some extent at the output start time. Therefore, in the state X2, the impedance Z_HF may be less likely to increase from the minimum value Zmin_HF as compared with the state X1 in which the treatment target has not been heat-denatured prior to the output command. In this case, contrary to the example of FIG. 5, the rate βb2 of increase in the state X2 is smaller than the rate βb1 of increase in the state X1. The rate βb1 of increase in the state X1 is equal to or greater than the reference value βbref, and the rate βb2 of increase in the state X2 is smaller than the reference value βbref. Thus, in accordance with the determination based on the rate βb of increase, the processor 25 appropriately determines whether or not the treatment target has been heat-denatured prior to receiving the output command, and appropriately sets the determination parameter η.

When the processor 25 performs a determination in accordance with any one of the patterns A4 to A6, the processor 25 determines the heat denaturation of the treatment target based on the variation with time in the impedance Z_HF after the detection of the minimum value Zmin_HF, that is, after the second phase terminates. Therefore, if it is determined that the treatment target has been heat-denatured prior to receiving the output command, that is, if the determination parameter η is set to the value η2, the processor 25 switches the operation state of the treatment instrument 2 from the first mode to the second mode in the middle of the third phase. When the processor 25 performs a determination in accordance with any one of the patterns A1 to A6, the processor 25 determines the heat denaturation of the treatment target based on a parameter associated with the impedance Z_HF. At this time, the parameter (Ze_HF; Ya; βa; Yb; βb) used for the determination is a parameter indicative of a reaction of the treatment target to the application of the treatment energy, that is, a parameter used for feedback control of the output of the first electrical energy.

In a seventh example, the processor 25 makes a determination of the heat denaturation in accordance with a pattern A7. In this case, based on a peak value Pp_HF of the output electric power P_HF, the processor 25 determines the heat denaturation of the treatment target and sets the determination parameter η. When the control for increasing the output voltage V_HF with time is performed as described above in the second phase, the output electric power P_HF increases with time from the initial value Pe_HF. When the output electric power P_HF increases to a certain extent, the output electric power P_HF changes from a state of increasing with time to a state of decreasing with time. As a result, the peak value Pp_HF of the output electric power P_HF is generated. The output electric power P_HF reaches the peak value Pp_HF at or immediately before or after a time when the impedance Z_HF reaches the minimum value Zmin_HF. In this example, if the peak value Pp_HF is greater than the reference value Ppref_HF, the processor 25 determines that the treatment target has not been heat-denatured prior to receiving the output command, and sets the determination parameter η to the value η1. On the other hand, if the peak value Pp_HF is equal to or smaller than the reference value Ppref_HF, the processor 25 determines that the treatment target has been heat-denatured prior to receiving the output command, and sets the determination parameter η to the value η2.

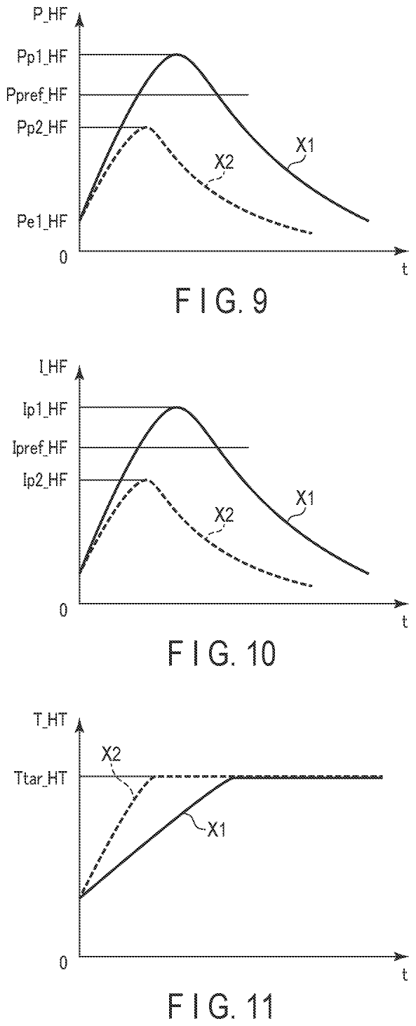

FIG. 9 shows an example of a variation with time in the output electric power P_HF in this example. In FIG. 9, the abscissa axis indicates time t with reference to the output start time, and the ordinate axis indicates the output electric power P_HF. In FIG. 9, the variation with time in the output electric power P_HF in the state X1 is indicated by the solid line, and the variation with time in the output electric power P_HF in the state X2 is indicated by the broken line. In the state X2 in which the treatment target has been heat-denatured prior to the output command, the initial value Ze_HF of the impedance Z_HF is greater and the high-frequency current is less likely to flow to the treatment target as compared with the state X1 in which the treatment target has not been heat-denatured prior to the output command. Therefore, in the state X2, the peak value Pp_HF of the output electric power P_HF is smaller than that in the state X1. For example, in the example of FIG. 9, a peak value Pp2_HF in the state X2 is smaller than a peak value Pp1_HF in the state X1. The peak value Pp1_HF in the state X1 is greater than the reference value Ppref_HF, and the peak value Pp2_HF in the state X2 is equal to or smaller than the reference value Ppref_HF. Therefore, in accordance with the determination based on the peak value Pp_HF, in the state X1, the processor 25 determines that the treatment target has not been heat-denatured prior to the output command. On the other hand, in the state X2, the processor 25 determines that the treatment target has been heat-denatured prior to the output command. Thus, the processor 25 appropriately determines whether or not the treatment target has been heat-denatured prior to receiving the output command, and appropriately sets the determination parameter η.

In an eighth example, the processor 25 makes a determination of the heat denaturation in accordance with a pattern A8. In this case, based on a peak value Ip_HF of the output current I_HF, the processor 25 determines the heat denaturation of the treatment target and sets the determination parameter η. When the control for increasing the output voltage V_HF with time is performed as described above in the second phase, the output current I_HF changes with time as in the case of the output electric power P_HF. Thus, the output current I_HF reaches the peak value Ip_HF at or immediately before or after a time when the impedance Z_HF reaches the minimum value Zmin_HF. In this example, if the peak value Ip_HF is greater than the reference value Ipref_HF, the processor 25 determines that the treatment target has not been heat-denatured prior to receiving the output command, and sets the determination parameter η to the value η1. On the other hand, if the peak value Ip_HF is equal to or smaller than the reference value Ipref_HF, the processor 25 determines that the treatment target has been heat-denatured prior to receiving the output command, and sets the determination parameter η to the value η2.

FIG. 10 shows an example of a variation with time in the output current I_HF in this example. In FIG. 10, the abscissa axis indicates time t, and the ordinate axis indicates the output current I_HF. In FIG. 10, the variation with time in the output current I_HF in the state X1 is indicated by the solid line, and the variation with time in the output current I_HF in the state X2 is indicated by the broken line. As described above, in the state X2 in which the treatment target has been heat-denatured prior to the output command, the high-frequency current is less likely to flow to the treatment target as compared with the state X1 in which the treatment target has not been heat-denatured prior to the output command. Therefore, in the state X2, the peak value Ip_HF of the output current I_HF is smaller than that in the state X1.

For example, in the example of FIG. 10, the peak value Ip2_HF in the state X2 is smaller than the peak value Ip1_HF in the state X1. The peak value Ip1_HF in the state X1 is greater than the reference value Ipref_HF, and the peak value Ip2_HF in the state X2 is equal to or smaller than the reference value Ppref_HF. Thus, in accordance with the determination based on the peak value Ip_HF, the processor 25 appropriately determines whether or not the treatment target has been heat-denatured prior to receiving the output command, and appropriately sets the determination parameter η.

In a ninth example, the processor 25 makes a determination of the heat denaturation in accordance with a pattern A9. In this case, based on an initial value Δφe of a phase difference Δφ between the output current I_HF and the output voltage V_HF, the processor 25 determines the heat denaturation of the treatment target and sets the determination parameter η. The initial value Δφe may be, for example, the phase difference Δφ at any point of time in the first phase, and may be an average value or an intermediate value of the phase difference Δφ in the first phase. In this example, if the absolute value of the initial value Δφe of the phase difference Δφ is smaller than the reference value Δφeref, the processor 25 determines that the treatment target has not been heat-denatured prior to receiving the output command, and sets the determination parameter η to the value η1. On the other hand, if the absolute value of the initial value Δφe is equal to or greater than the reference value Δφeref, the processor 25 determines that the treatment target has been heat-denatured prior to receiving the output command, and sets the determination parameter η to the value η2.

In the state X2 in which the treatment target has been heat-denatured prior to the output command, the treatment target is already dried to some extent. Therefore, the capacitance component of the impedance Z_HF increases, and the output current I_HF advances over the output voltage V_HF. On the other hand, in the state X1 in which the treatment target has not been heat-denatured prior to the output command, the output current I_HF is not greatly shifted from the output voltage V_HF immediately after the output start. Therefore, in the state X2 in which the treatment target has been heat-denatured prior to the output command, the absolute value of the initial value Δφe of the phase difference Δφ is greater than that in the state X1 in which the treatment target has not been heat-denatured prior to the output command. In one example, the absolute value of the initial value Δφe2 in state X2 is greater than the absolute value of the initial value Δφe1 in state X1. The absolute value of the initial value Δφe1 in the state X1 is smaller than the reference value Δφeref, and the absolute value of the initial value Δφe2 in the state X2 is equal to or greater than the reference value Δφeref. Therefore, in accordance with the determination based on the initial value Δφe, the processor 25 appropriately determines whether or not the treatment target has been heat-denatured prior to receiving the output command, and appropriately sets the determination parameter η.

In a tenth example, the processor 25 makes a determination of the heat denaturation in accordance with a pattern A10. In this case, based on an initial value Δφe of a phase difference Δφ, the processor 25 also determines the heat denaturation of the treatment target and sets the determination parameter η. However, in this example, if the initial value Δφe of the phase difference Δφ is 0 or smaller, that is, if the initial value Δφe is 0 or a negative value, the processor 25 determines that the treatment target has not been heat-denatured prior to receiving the output command, and sets the determination parameter η to the value η1. On the other hand, if the initial value Δφe is a positive value, the processor 25 determines that the treatment target has been heat-denatured prior to receiving the output command, and sets the determination parameter η to the value η2.

In a state X1 in which the treatment target has not been heat-denatured prior to the output command, since the treatment target contains a large amount of moisture, the inductance components of the impedance Z_HF become large, and the output current I_HF is slightly delayed from the output voltage V_HF. Therefore, in the state X1, the initial value Δφe of the phase difference Δφ becomes a negative value. On the other hand, in a state X2 in which the treatment target has been heat-denatured prior to the output command, the initial value Δφe becomes a positive value because the output current I_HF advances over the output voltage V_HF as described above. Therefore, in accordance with the determination based on the initial value Δφe, in the state X1, the processor 25 determines that the treatment target has not been heat-denatured prior to the output command. On the other hand, in the state X2, the processor 25 determines that the treatment target has been heat-denatured prior to the output command. Thus, the processor 25 appropriately determines whether or not the treatment target has been heat-denatured prior to receiving the output command, and appropriately sets the determination parameter η.

When the processor 25 makes a determination in accordance with any of the patterns A1 to A10, the processor 25 determines the heat denaturation of the treatment target based on a parameter associated with the first electrical energy output to the electrodes 21 and 22. At this time, the parameter (Ze_HF; Ya; βa; Yb; βb; Pp_HF; Ip_HF; Δφe) used for the determination is a parameter indicative of a reaction of the treatment target to the application of the treatment energy, and a parameter used for feedback control of the output of the first electrical energy. Further, in the treatment system 1 in which the heater 23 is not provided and the electric power source device 3 outputs electrical energy (first electrical energy) only to the electrodes 21 and 22, the processor 25 can make a determination in accordance with any of the patterns A1 to A10.

In an eleventh example, the processor 25 makes a determination of the heat denaturation in accordance with a pattern A11. In this case, based on a rate γa of increase in the temperature T_HT of the heater 23 until the temperature reaches the set target temperature Ttar_HT, the processor 25 determines the heat denaturation of the treatment target and sets the determination parameter η. In this example, if the rate γa of increase is smaller than the reference value γaref, the processor 25 determines that the treatment target has not been heat-denatured prior to receiving the output command, and sets the determination parameter η to the value η1. On the other hand, if the rate γa of increase is equal to or greater than the reference value γaref, the processor 25 determines that the treatment target has been heat-denatured prior to receiving the output command, and sets the determination parameter η to the value η2.

FIG. 11 shows an example of a variation with time in the temperature T_HT of the heater 23 according to the present example. In FIG. 11, the abscissa axis indicates time t, and the ordinate axis indicates the temperature T_HT. In FIG. 11, the variation with time in the temperature T_HT in the state X1 is indicated by the solid line, and the variation with time in the temperature T_HT in the state X2 is indicated by the broken line. In the state X2 in which the treatment target has been heat-denatured prior to the output command, the heat load of the treatment target is small, and the temperature of the treatment target is easily increased. Therefore, in the state X2, the rate γa of increase in the temperature T_HT is greater than in the state X1 in which the treatment target has not been heat-denatured prior to the output command. For example, in an example of FIG. 11, the rate γa2 of increase in the state X2 is greater than the rate γa1 of increase in the state X1. The rate γa1 of increase in the state X1 is smaller than the reference value γaref, and the rate γa2 of increase in the state X2 is equal to or greater than the reference value γaref. Therefore, in accordance with the determination based on the rate γa of increase, in the state X1, the processor 25 determines that the treatment target has not been heat-denatured prior to the output command. On the other hand, in the state X2, the processor 25 determines that the treatment target has been heat-denatured prior to the output command. Thus, the processor 25 appropriately determines whether or not the treatment target has been heat-denatured prior to receiving the output command, and appropriately sets the determination parameter η.

When the processor 25 makes a determination in accordance with the pattern A11, the processor 25 determines the heat denaturation of the treatment target based on a parameter associated with the temperature T_HT of the heater 23, and related to the second electrical energy output to the heater 23. At this time, the parameter (γa) used for the determination is a parameter indicative of a reaction of the treatment target to the application of the treatment energy, and a parameter used for feedback control of the output of the second electrical energy. In the treatment system 1 in which the electrodes 21 and 22 are not provided and electrical energy (second electrical energy) is output only to the heater 23 from the electric power source device 3, the processor 25 can also make a determination in accordance with the pattern A11. When the processor 25 makes a determination in accordance with any of the patterns A1 to A11, the processor 25 determines the heat denaturation of the treatment target based on parameters associated with the electrical energy supplied to the treatment instrument 2, including the first electrical energy and the second electrical energy.

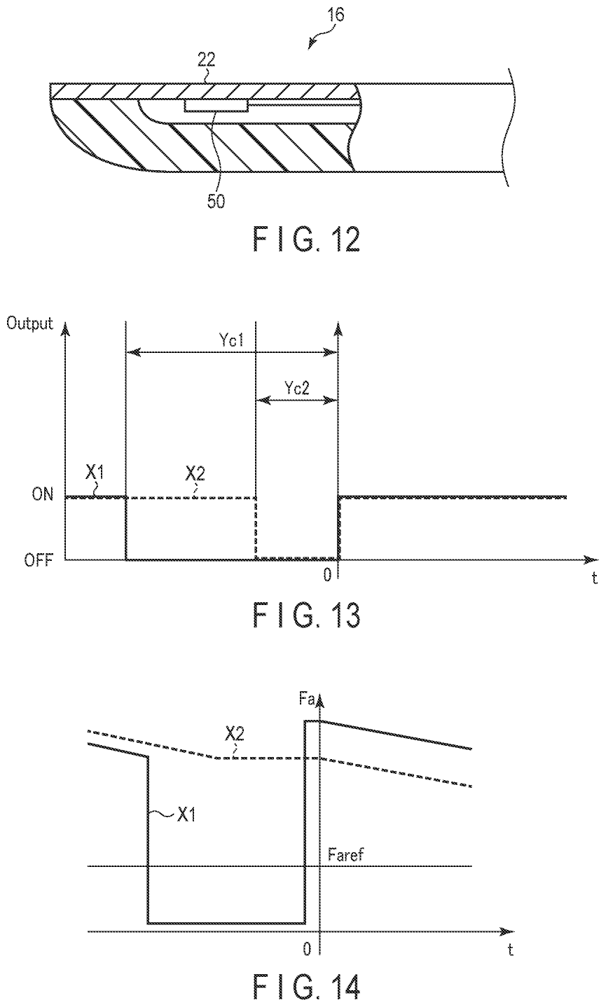

In a twelfth example, the processor 25 makes a determination of the heat denaturation in accordance with a pattern A12. FIG. 12 shows a configuration of one of the grasping pieces 15 and 16 of the present embodiment. As shown in FIG. 12, a temperature sensor 50 is attached to the electrode 22 of the grasping piece 16 in this example. The temperature sensor 50 detects the temperature T_S of the treatment target at or immediately before the output start time of the electrical energy (t=0). The processor 25 acquires a detection result of the temperature T_S at the temperature sensor 50. In this example, based on the acquired temperature T_S, the processor 25 determines the heat denaturation of the treatment target and sets the determination parameter η. The temperature T_S of the treatment target (tissue) is a parameter indicative of a reaction of the treatment target to the application of treatment energy. In this example, if the temperature T_S is lower than the reference value Tref_S, the processor 25 determines that the treatment target has not been heat-denatured prior to receiving the output command, and sets the determination parameter η to the value η1. On the other hand, if the temperature T_S is equal to or higher than the reference value Tref_S, the processor 25 determines that the treatment target has been heat-denatured prior to receiving the output command, and sets the determination parameter η to the value η2. The reference value Tref_S is, for example, 60° C.

In the state X2 in which the treatment target has been heat-denatured prior to the output command, the temperature T_S of the treatment target has already increased to some extent. Therefore, in the state X2, the temperature T_S of the treatment target is higher at and immediately before the output start time than in the state X1 in which the treatment target has not been heat-denatured prior to the output command. The temperature T1_S in the state X1 is lower than the reference value Tref_S, and the temperature T2_S in the state X2 is equal to or higher than the reference value Tref_S. In this example, therefore, in accordance with the determination based on the temperature T_S, the processor 25 appropriately determines whether or not the treatment target has been heat-denatured prior to receiving the output command, and appropriately sets the determination parameter η.

In one example, an observation device (not shown), such as an endoscope, is used to observe the treatment target and the end effector. The processor 25 acquires an observation image observed by the observation device. In this case, based on a parameter acquired from the observation image, for example, a luminance of the treatment target, the processor 25 determines the heat denaturation of the treatment target and sets the determination parameter η. When the treatment target is heat-denatured by the application of the treatment energy, the treatment target becomes white and the luminance of the treatment target becomes high in the observation image. Thus, the luminance of the treatment target is a parameter indicative of a reaction of the treatment target to the application of the treatment energy. In this example, if the luminance is smaller than the reference value, the processor 25 determines that the treatment target has not been heat-denatured prior to receiving the output command, and sets the determination parameter η to the value η1. On the other hand, if luminance is equal to or greater than the reference value, the processor 25 determines that the treatment target has been heat-denatured prior to receiving the output command, and sets the determination parameter η to the value η2. In this case, similarly to the examples described above, the processor 25 appropriately determines whether or not the treatment target has been heat-denatured prior to receiving the output command, and appropriately sets the determination parameter η.

In a thirteenth example, the processor 25 makes a determination of the heat denaturation in accordance with a pattern A13. In this case, based on time Yc elapsed from the termination of the previous output until the start of output of the electrical energy (t=0), the processor 25 determines the heat denaturation of the treatment target and sets the determination parameter η. In this example, if the time Yc is longer than a reference time Ycref, the processor 25 determines that the treatment target has not been heat-denatured prior to receiving the output command, and sets the determination parameter η to the value η1. On the other hand, if the time Yc is equal to or shorter than the reference time Ycref, the processor 25 determines that the treatment target has been heat-denatured prior to receiving the output command, and sets the determination parameter η to the value η2.

FIG. 13 shows an example of a variation with time in ON-OFF states of an output of electrical energy to the treatment instrument 2 in this example. In FIG. 13, the abscissa axis indicates time t (t=0 at the output start time), and the ordinate axis indicates ON-OFF states of the output of the electrical energy. In FIG. 13, the variation with time in the ON-OFF states in the state X1 is indicated by the solid line, and the variation with time in the ON-OFF states in the state X2 is indicated by the broken line. In the state X1 in which the treatment energy is applied for the first time to the treatment target which has not been heat-denatured prior to the output command, the end effector 7 is moved to the treatment target from the position where the treatment energy is applied by the previous output, and the output of the electrical energy is subsequently started. On the other hand, in the state X2 in which the treatment energy is again applied to the portion which has already been heat-denatured by the previous output, the output of the electrical energy is started without moving the end effector 7 from the previous output. Therefore, in the state X2, the time Yc elapsed from the termination of the previous output is shorter than in the state X1. For example, in the example of FIG. 13, the time Yc2 in the state X2 is shorter than the time Yc1 in the state X1. The time Yc1 in the state X1 is longer than the reference time Ycref, and the time Yc2 in the state X2 is equal to or shorter than the reference time Ycref. Therefore, in accordance with the determination based on the reference time Yc, the processor 25 appropriately determines whether or not the treatment target has been heat-denatured prior to receiving the output command, and appropriately sets the determination parameter η.

In a fourteenth example, the processor 25 makes a determination of the heat denaturation in accordance with a pattern A14. In this example, a pressure sensor (not shown) is attached to one of the grasping pieces 15 and 16. The pressure sensor detects a pressing force Fa applied to the end effector 7 (one of the grasping pieces 15 and 16) from the tissue including the treatment target in at least a period from the termination of the previous output to the start of the current output (t=0). The processor 25 acquires the detection result of the pressing force Fa in the pressure sensor. In this example, based on the acquired pressing force Fa, the processor 25 determines the heat denaturation of the treatment target and sets the determination parameter η. In this example, if the pressing force Fa changes to a value smaller than a reference value Faref in a period from the termination of the previous output to the start of the current output, the processor 25 determines that the treatment target has not been heat-denatured prior to receiving the output command, and sets the determination parameter η to the value η1. On the other hand, if the pressing force Fa is continuously maintained to a value equal to or greater than the reference value Faref in the period from the termination of the previous output to the start of the current output, the processor 25 determines that the treatment target has been heat-denatured prior to receiving the output command, and sets the determination parameter η to the value η2.

FIG. 14 shows an example of a variation with time in the pressing force Fa from a tissue to the end effector 7 of the example. In FIG. 14, the abscissa axis indicates time t (t=0 at the output start time), and the ordinate axis indicates the pressing force Fa. In FIG. 14, the variation with time in the pressing force Fa in the state X1 is indicated by the solid line, and the variation with time in the pressing force Fa in the state X2 is indicated by the broken line. In the state X1, in which the treatment energy is applied for the first time to the treatment target which has not been heat-denatured prior to the output command, the end effector 7 is moved to the treatment target from the position where the previous output is performed as described above, and the output of the electrical energy is started. Therefore, it is necessary to move the end effector 7 by opening the grasping pieces 15 and 16 relative to each other from the termination of the previous output. By opening the grasping pieces 15 and 16, the pressing force Fa temporarily decreases to a value close to 0, and temporarily changes to a value smaller than the reference value Faref in a period from the termination of the previous output to the start of the current output. On the other hand, in the state X2 in which the treatment energy is again applied to the portion which has already been heat-denatured by the previous output, as described above, the output of the electrical energy is started without moving the end effector 7 from the portion where the previous output is performed. Therefore, the grasping pieces 15 and 16 are maintained in a closed state from the termination of the previous output. By maintaining the grasping pieces 15 and 16 in the closed state, the pressing force Fa is maintained at the reference value Faref or greater in a period from the termination of the previous output to the start of the current output. For example, as shown in FIG. 14, in the state X1, the pressing force Fa is temporarily decreased below the reference value Faref, whereas in the state X2, the pressing force Fa is continuously maintained to be equal to or greater than the reference value Faref. Therefore, in accordance with the determination based on the pressing force Fa, the processor appropriately determines whether or not the treatment target has been heat-denatured prior to receiving the output command, and appropriately sets the determination parameter η.

In a fifteenth example, the processor 25 makes a determination of the heat denaturation in accordance with a pattern A15. In this example, a gap sensor (not shown) is attached to one of the grasping pieces 15 and 16. The gap sensor detects a gap (distance) G between the grasping pieces 15 and 16 at least in a period from the termination of the previous output to the start of the current output. The processor 25 acquires the result of detection of the gap G by the gap sensor. In this example, based on the detected gap G, the processor 25 determines the heat denaturation of the treatment target and sets the determination parameter η. In this example, if the gap G changes to a value greater than a reference value Gref in a period from the termination of the previous output to the start of the current output, the processor 25 determines that the treatment target has not been heat-denatured prior to receiving the output command, and sets the determination parameter η to the value η1. On the other hand, if the gap G is continuously maintained to a value equal to or smaller than the reference value Gref in the period from the termination of the previous output to the start of the current output, the processor 25 determines that the treatment target has been heat-denatured prior to receiving the output command, and sets the determination parameter η to the value η2.

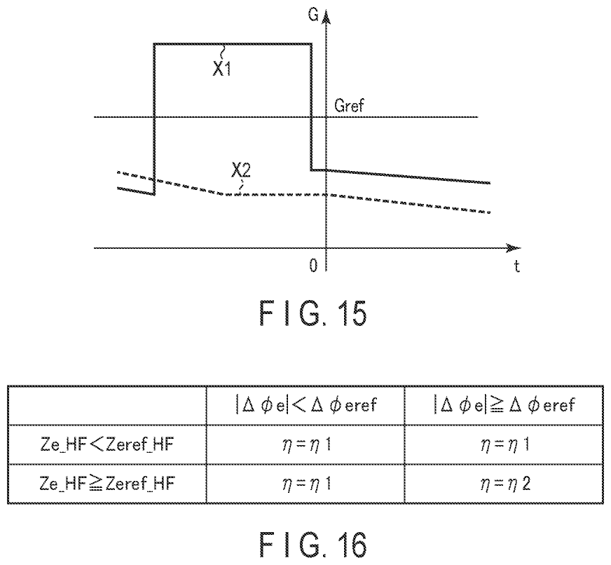

FIG. 15 shows an example of a variation with time in the gap G between the grasping pieces 15 and 16 in this example. In FIG. 15, the abscissa axis indicates time t, and the ordinate axis indicates the gap G. In FIG. 15, the variation with time in the gap G in the state X1 is indicated by the solid line, and the variation with time in the gap G in the state X2 is indicated by the broken line. In the state X1 in which the treatment energy is applied for the first time to the treatment target which has not been heat-denatured prior to the output command, as described above, the grasping pieces 15 and 16 are opened relative to each other from the termination of the previous output, thus moving the end effector 7. Therefore, the gap G temporarily changes to a value greater than the reference value Gref in a period from the termination of the previous output to the start of the current output. On the other hand, in the state X2 in which the treatment energy is again applied to the portion which has already been heat-denatured by the previous output, the grasping pieces 15 and 16 are maintained in a closed state from the termination of the previous output, as described above. Therefore, the gap G is maintained to a value equal to or smaller than the reference value Gref in a period from the termination of the previous output to the start of the current output. For example, as shown in FIG. 15, in the state X1, the gap G is temporarily increased above the reference value Gref, whereas in the state X2, the gap G is continuously maintained to be equal to or smaller than the reference value Gref. Therefore, in accordance with the determination based on the gap G, the processor 25 appropriately determines whether or not the treatment target has been heat-denatured prior to receiving the output command, and appropriately sets the determination parameter η.

In one example, the processor 25 determines whether or not the grasping pieces 15 and 16 are opened relative to each other in a period from the termination of the previous output to the start of the current output, based on an observation image of an observation device such as an endoscope. Based on the result of the determination on the opening and closing of the grasping pieces 15 and 16, the processor 25 appropriately determines whether or not the treatment target has been heat-denatured prior to receiving the output command, and appropriately sets the determination parameter η.

Furthermore, in one example, a plurality of parameters are acquired from the parameters described above, and the heat denaturation of the treatment target is determined based on the acquired parameters. For example, in a sixteenth example, the processor 25 determines, based on the initial value Ze_HF of the impedance Z_HF and the initial value Δφe of the phase difference Δφ, whether the treatment target has been heat-denatured prior to receiving the output command. FIG. 16 illustrates a setting of a determination parameter η by the processor 25 in this example. As shown in FIG. 16, in this example, if the initial value Ze_HF is smaller than the reference value Zeref_HF, or if the absolute value of the initial value Δφe of the phase difference Δφ is smaller than the reference value Δφeref, the processor 25 determines that the treatment target has not been heat-denatured prior to receiving the output command, and sets the determination parameter η to the value η1. On the other hand, if the initial value Ze_HF is equal to or greater than the reference value Zeref_HF and the absolute value of the initial value Δφe is equal to or greater than the reference value Δφeref, the processor 25 determines that the treatment target has been heat-denatured prior to receiving the output command, and sets the determination parameter η to the value η2.

In another example, based on the initial value Ze_HF of the impedance Z_HF and the rate γa of increase in the temperature T_HT of the heater 23 to the target temperature Ttar_HT, the processor 25 determines whether the treatment target has been heat-denatured prior to receiving the output command. In accordance with a determination based on the plurality of parameters, the processor 25 more appropriately determines whether or not the treatment target has been heat-denatured prior to receiving an output command.

[Pattern of Output Control on Electrical energy Based on Result of Determination of Heat Denaturation]

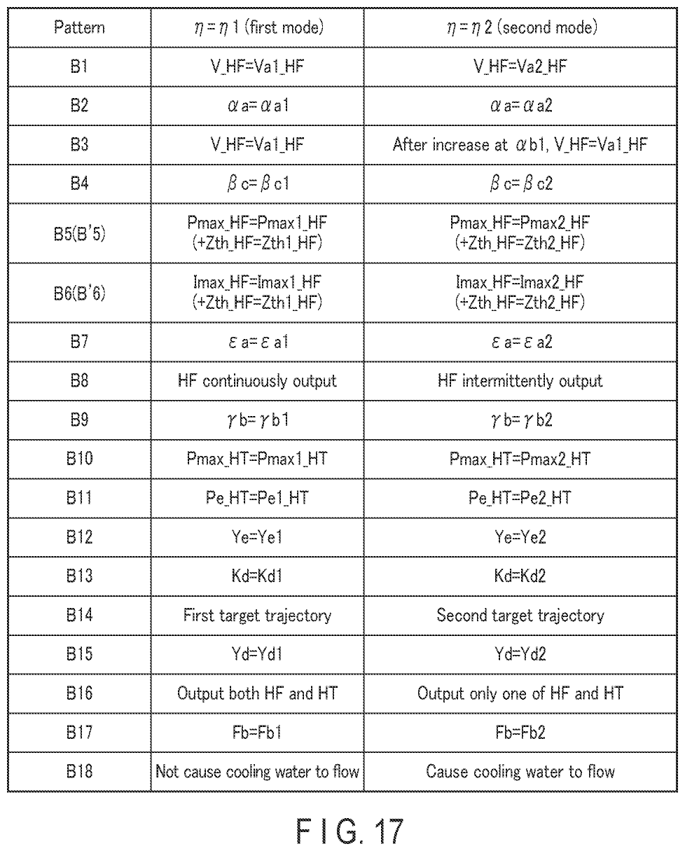

In the first example shown in FIGS. 4 to 7, in the constant voltage control of the first electrical energy in the third phase, the processor 25 sets the target value Va_HF of the output voltage V_HF to differing values for the first mode and the second mode. This causes the processor 25 to operate the treatment instrument 2 in differing states in the first mode and the second mode. However, in another example, the processor 25 may set the setting value other than the target value Va_HF in the second mode to a value different from that in the first mode. FIG. 17 shows a pattern of an output control on electrical energy by the processor 25 based on a result of determination of heat denaturation of the treatment target. In the first example, as described above, the processor 25 controls the output of the electrical energy based on the result of the determination of the heat denaturation in accordance with a pattern B1.

In a seventeenth example, the processor 25 performs the output control on the electrical energy based on the result of the determination of the heat denaturation in accordance with a pattern B2. In this case, during the control to increase the output voltage V_HF with time in the second phase, the processor 25 sets a rate aa of increase (setting value) in the output voltage V_HF to differing values for the first mode and the second mode. That is, the processor 25 sets the rate aa of increase, which is a variation rate with time in the output voltage V_HF, to differing values for the first mode and the second mode. This causes the processor 25 to operate the treatment instrument 2 in states different from each other in the first mode and the second mode. In this example, if the determination parameter η is set to the value η1, that is, if the treatment instrument 2 is operated in the first mode, the processor 25 sets the rate αa1 of increase (setting value). On the other hand, if the determination parameter η is set to the value η2, that is, if the treatment instrument 2 is operated in the second mode, the processor 25 sets the rate αa2 of increase (setting value) that is smaller than the rate αa1 of increase. Thus, in the second mode, the processor 25 suppresses the output of the first electrical energy from the electric power source device (generator) 3 to the treatment instrument 2 as compared with the first mode. In the third phase, in both the first mode and the second mode, the processor 25 performs the constant voltage control on the output voltage V_HF at the target value Va1_HF. In this example, if it is determined that the treatment target has been heat-denatured prior to receiving the output command, that is, if the determination parameter η is set to the value η2, the processor 25 switches the operation state of the treatment instrument 2 from the first mode to the second mode either at the start or in the middle of the second phase.

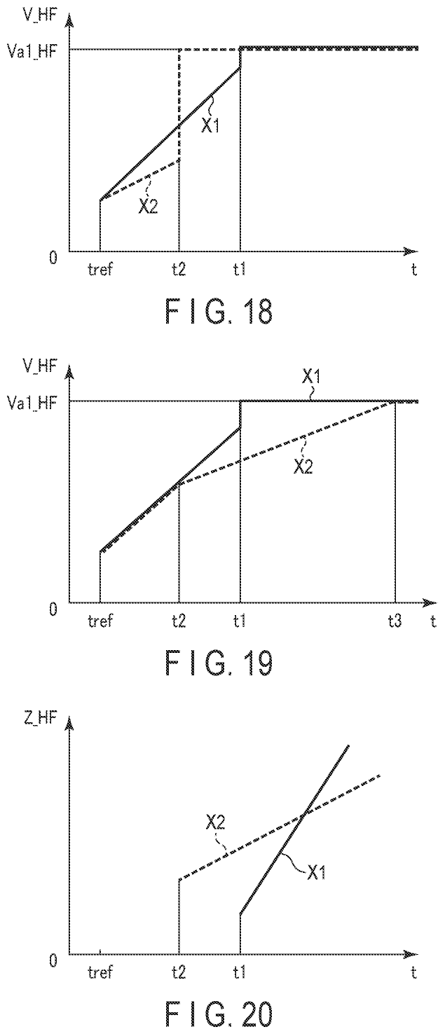

FIG. 18 shows an example of a target trajectory of the output voltage V_HF in this example. In FIG. 18, the abscissa axis indicates time t, and the ordinate axis indicates the output voltage V_HF. FIG. 18 shows target trajectories of the output voltage V_HF in the second phase and the third phase, and the target trajectory of the output voltage V_HF in the state X1 is indicated by the solid line, while the target trajectory of the output voltage V_HF in the state X2 is indicated by the broken line. As shown in FIG. 18, the processor 25 of the present example performs the constant voltage control to maintain the output voltage V_HF at the target value Va1_HF in the third phase in both the states X1 and X2. However, in the state X1 in which the determination parameter η is set to the value η1, the processor 25 performs the control to increase the output voltage V_HF with time at the rate αa1 of increase in the second phase, and causes the treatment instrument to operate in the first mode. On the other hand, in the state X2 in which the determination parameter η is set to the value η2, the processor 25 performs the control to increase the output voltage V_HF with time at the rate αa2 of increase, which is smaller than the rate αa1 of increase, in the second phase, and causes the treatment instrument to operate in the second mode. Thus, in the state X2, the output of the first electrical energy to the electrodes 21 and 22 is suppressed and the application of the high-frequency current (treatment energy) to the treatment target is suppressed in the second phase as compared with the state X1. Accordingly, also in this example, in the state X2 in which the treatment energy is applied second and subsequent times to the already heat-denatured portion, the appropriate treatment performance can be exhibited.

In an eighteenth example, the processor 25 performs the output control on the electrical energy based on the result of the determination of the heat denaturation in accordance with a pattern B3. In this case, the processor 25 sets the target trajectory (setting trajectory) of the output voltage V_HF in the third phase to trajectories different from each other for the first mode and the second mode. That is, the processor 25 sets the target value of the output voltage V_HF in each time t in the third phase to values different from each other for the first mode and the second mode. By setting the target trajectory of the output voltage V_HF in the second mode to a trajectory different from that of the first mode, the processor 25 causes the treatment instrument 2 to operate in states different from each other in the first mode and the second mode. In this example, if the determination parameter η is set to the value η1, the processor 25 performs the constant voltage control on the output voltage V_HF at the target value Va1_HF in the third phase. On the other hand, if the determination parameter η is set to the value η2, the processor 25 increases the output voltage V_HF with time at the rate αb1 of increase from the start of the third phase. At this time, the processor 25 sets the rate αb1 of increase to a value smaller than the rate αa1 of increase in the output voltage V_HF in the second phase. After the output voltage V_HF is increased to the voltage value Va1_HF, the processor 25 performs the constant voltage control on the output voltage V_HF at the target value Va1_HF. By performing the control in the third phase as described above, in the second mode, the processor 25 suppresses the output of the first electrical energy from the electric power source device (generator) 3 to the treatment instrument 2 as compared with the first mode.

FIG. 19 shows an example of the target trajectory of the output voltage V_HF in this example. In FIG. 19, the abscissa axis indicates time t, and the ordinate axis indicates the output voltage V_HF. FIG. 19 shows target trajectories of the output voltage V_HF in the second phase and the third phase, and the target trajectory of the output voltage V_HF in the state X1 is indicated by a solid line, while the target trajectory of the output voltage V_HF in the state X2 is indicated by a broken line. As shown in FIG. 19, the processor 25 of the present example performs the control to increase the output voltage V_HF at the rate αa1 of increase in the second phase in both the states X1 and X2. However, in the state X1, the processor 25 performs the constant voltage control on the output voltage V_HF at the target value Va1_HF from the start of the third phase (t=t1). On the other hand, in the state X2, the processor 25 performs the control to increase the output voltage V_HF with time at the rate αb1 of increase for a while from the start of the third phase (t=t2). At the time t3 after the start of the third phase, the processor 25 starts the constant voltage control on the output voltage V_HF at the target value Va1_HF. Thus, in the state X2, the output of the first electrical energy to the electrodes 21 and 22 is suppressed and the application of the high-frequency current to the treatment target is suppressed in the third phase as compared with the state X1. Accordingly, also in this example, the appropriate treatment performance can be exhibited in the state X2.

In a nineteenth example, the processor 25 performs the output control on the electrical energy based on the result of the determination of the heat denaturation in accordance with a pattern B4. In this case, the processor 25 sets a rate βc of increase (setting value) with time in the impedance Z_HF, and sets a target trajectory of the impedance Z_HF which increases with time at the set rate βc of increase. In the third phase, the processor 25 performs the output control on the first electrical energy to increase the impedance Z_HF with time at the rate βc of increase. In this example, the processor 25 sets the rate βc of increase in the impedance Z_HF in the third phase to differing values for the first mode and the second mode. That is, the processor 25 sets the rate βc of increase, which is a variation rate with time in the impedance Z_HF, to differing values for the first mode and the second mode. As a result, the processor 25 sets the target trajectory (setting trajectory) of the impedance Z_HF in the third phase to trajectories different from each other for the first mode and the second mode. That is, the processor 25 sets the target value of the impedance Z_HF in each time t in the third phase to differing values for the first mode and the second mode. By setting the rate βc of increase in the impedance Z_HF in the second mode to a value different from that of the first mode, the processor 25 causes the treatment instrument 2 to operate in states different from each other in the first mode and the second mode.

In this example, if the determination parameter η is set to the value η1, the processor 25 performs the output control on the first electrical energy to increase the impedance Z_HF with time at the rate βc1 of increase in the third phase. On the other hand, if the determination parameter η is set to the value η2, the processor 25 performs the output control on the first electrical energy to increase the impedance Z_HF with time at the rate βc2 of increase, which is smaller than the rate βc1 of increase. Thus, in the second mode, the processor 25 increases the impedance Z_HF more gradually in the third phase and suppresses the output voltage V_HF in the third phase as compared with the first mode. Thus, in the second mode, the processor 25 suppresses the output of the first electrical energy from the electric power source device (generator) 3 to the treatment instrument 2 as compared with the first mode.

FIG. 20 shows an example of the target trajectory of the impedance Z_HF in this example. In FIG. 20, the abscissa axis indicates time t, and the ordinate axis indicates the impedance Z_HF. FIG. 20 shows target trajectories of the impedance Z_HF in the third phase, and the target trajectory of the impedance Z_HF in the state X1 is indicated by the solid line, while the target trajectory of the impedance Z_HF in the state X2 is indicated by the broken line. As shown in FIG. 20, the processor 25 of the present example performs the control to increase the impedance Z_HF at the rate βc1 of increase in the third phase in the state X1. On the other hand, in the state X2, the processor 25 performs the control to increase the impedance Z_HF at the rate βc2 of increase, which is smaller than the rate βc1 of increase, in the third phase. Thus, in the state X2, the output voltage V_HF in the third phase is suppressed and the output of the first electrical energy to the electrodes 21 and 22 is suppressed in the third phase as compared with the state X1. Therefore, in the state X2, the application of the high-frequency current to the treatment target is suppressed in the third phase as compared with the state X1. Accordingly, also in this example, in the state X2, the appropriate treatment performance can be exhibited.

In a twentieth example, the processor 25 performs the output control on the electrical energy based on the result of the determination of the heat denaturation in accordance with a pattern B5. In this case, the processor 25 sets the upper limit value (setting value) Pmax_HF of an outputtable range of the output electric power P_HF to differing values for the first and second modes. This causes the processor to operate the treatment instrument 2 in states different from each other in the first mode and the second mode. In this example, if the determination parameter η is set to the value η1, the processor 25 sets the upper limit value Pmax1_HF for the outputtable range of the output electric power P_HF. On the other hand, if the determination parameter η is set to the value η2, the processor 25 sets the upper limit value Pmax2_HF, which is smaller than the upper limit value Pmax1_HF, for the outputtable range of the output electric power P_HF. Thus, in the second mode, the processor 25 suppresses the output of the first electrical energy from the electric power source device (generator) 3 to the treatment instrument 2 as compared with the first mode. In this example, if it is determined that the treatment target has been heat-denatured prior to receiving the output command, the processor 25 switches the operation state of the treatment instrument 2 from the first mode to the second mode either at the start or in the middle of the second phase.

In a twenty-first example, the processor 25 performs the output control on the electrical energy based on the result of the determination of the heat denaturation in accordance with a pattern B'S. In this case, in the same manner as in the twentieth example, the processor 25 sets the upper limit value Pmax_HF of an outputtable range of the output electric power P_HF. Further, in this example, the processor 25 sets, in addition to the upper limit value Pmax_HF, a threshold value Zth_HF of the impedance Z_HF associated with the termination condition of the output of the first electrical energy to differing values for the first mode and the second mode. If the determination parameter η is set to the value η1, the processor 25 sets a threshold value Zth1_HF as the setting value associated with the termination condition. On the other hand, if the determination parameter η is set to the value η2, the processor 25 sets a threshold value Zth2_HF, which is greater than the threshold value Zth1_HF, as the setting value associated with the termination condition.

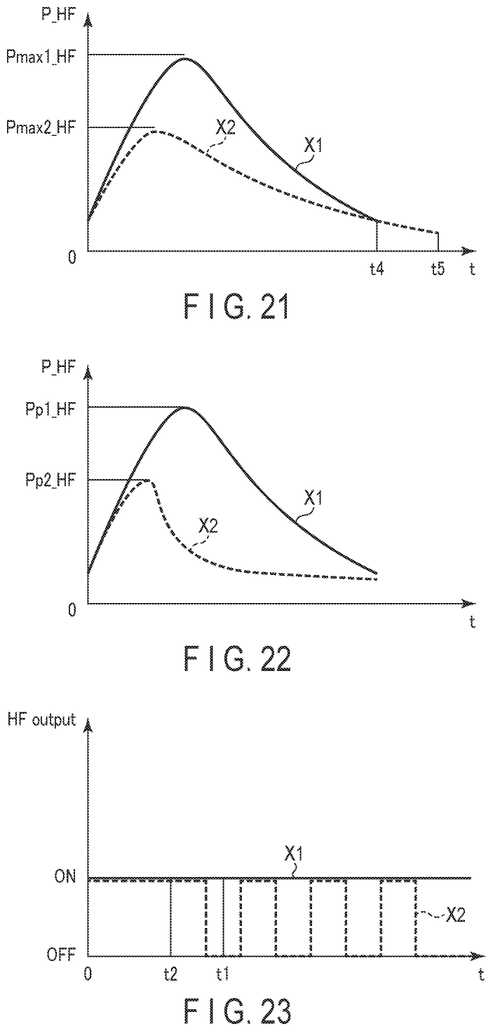

FIG. 21 shows an example of a variation with time in the output electric power P_HF in this example. In FIG. 21, the abscissa axis indicates time t, and the ordinate axis indicates electric power P_HF. In FIG. 21, the variation with time in the output electric power P_HF in the state X1 is indicated by the solid line, and the variation with time in the output electric power P_HF in the state X2 is indicated by the broken line. As shown in FIG. 21, in the state X1, the processor 25 causes the output electric power P_HF to be output below the upper limit value Pmax1_HF. On the other hand, in the state X2, the processor 25 causes the output electric power P_HF to be output below the upper limit value Pmax2_HF, which is smaller than the upper limit value Pmax1_HF. Thus, in the state X2, the output electric power P_HF from the output source 31 is suppressed and the output of the first electrical energy to the electrodes 21 and 22 is suppressed as compared with the state X1. Therefore, in the state X2, the application of the high-frequency current to the treatment target is suppressed as compared with the state X1. Accordingly, also in this example, in the state X2, the appropriate treatment performance can be exhibited. This also applies to the twentieth example.

In this example, in the state X1, the processor 25 sets the threshold value Zth1_HF as the setting value associated with the termination condition. In the state X2, the processor 25 sets the threshold value Zth2_HF, which is greater than the threshold value Zth1_HF, as the setting value associated with the termination condition. Therefore, in the state X1, the processor 25 determines at time t4 that the impedance Z_HF has reached the threshold value Zth1_HF, and ceases the output of the electrical energy (first electrical energy) at time t4. On the other hand, in the state X2, the processor 25 determines at time t5 later than time t4 that the impedance Z_HF has reached the threshold value Zth2_HF, and ceases the output of the electrical energy at time t5. Therefore, in the state X2 in which the treatment target has been heat-denatured prior to the output command, the processor 25 increases the time for applying the treatment energy to the treatment target as compared with the state X1 in which the treatment target has not been heat-denatured prior to the output command. Accordingly, in the state X2, the processor 25 suppresses the treatment energy applied to the treatment target and increases the time for applying the treatment energy to the treatment target as compared with the state X1.

In a twenty-second example, the processor 25 performs the output control on the electrical energy based on the result of the determination of the heat denaturation in accordance with a pattern B6. In this case, the processor 25 sets the upper limit value (setting value) Imax_HF of an outputtable range of the output current I_HF to differing values for the first and second modes. This causes the processor 25 to operate the treatment instrument 2 in states different from each other in the first mode and the second mode. In this example, if the determination parameter η is set to the value η1, the processor 25 sets the upper limit value Imax1_HF for the outputtable range of the output current I_HF. On the other hand, if the determination parameter η is set to the value η2, the processor 25 sets the upper limit value Imax2_HF, which is smaller than the upper limit value Imax1_HF, for the outputtable range of the output electric power P_HF. Thus, in the second mode, the processor 25 suppresses the output of the first electrical energy from the electric power source device (generator) 3 to the treatment instrument 2 as compared with the first mode.

In a twenty-third example, the processor 25 performs the output control on the electrical energy based on the result of the determination of the heat denaturation in accordance with a pattern B′6. In this case, in the same manner as in the twenty-second example, the processor 25 sets the upper limit value Imax_HF of an outputtable range of the output current I_HF. Further, in this example, the processor 25 sets, in addition to the upper limit value Imax_HF, a threshold value Zth_HF of the impedance Z_HF associated with the termination condition of the output of the electrical energy to differing values for the first mode and the second mode. In this example, the processor 25 sets the threshold value Zth_HF in the same manner as in the twenty-first example.

In the twenty-second and twenty-third examples, as in the twentieth and twenty-first examples, the processor 25 suppresses the output of the first electrical energy to the treatment instrument 2 in the state X2 as compared with the state X1. Therefore, in the twenty-second and twenty-third examples, in the state X2, the appropriate treatment performance can be exhibited. In the twenty-third example, as in the twenty-first example, in the state X2, the processor 25 increases the time for applying the treatment energy to the treatment target as compared with the state X1.

In one example, the processor 25 may set a termination condition of the output of the electrical energy based on a parameter other than the impedance Z_HF. In this case, the processor 25 sets a threshold value Δφth of a phase difference Δφ as a setting value associated with the termination condition. The processor 25 ceases the output of the electrical energy based on the phase difference Δφ reaching the threshold value Δφth. Further, if a threshold value Δφth of the phase difference Δφ is set, the processor 25 may set, in addition to the upper limit value Pmax_HF or the upper limit value Imax_HF, a threshold value Δφth of the phase difference Δφ associated with the termination condition of the output of the electrical energy to differing values for the first mode and the second mode, in the same manner as the threshold value Zth_HF of the twenty-first and the twenty-third examples.

In another example, the processor 25 sets a threshold value Ydth for a time Yd from the start of the third phase as a setting value associated with the termination condition. Then, the processor 25 ceases the output of the electrical energy based on the fact that the time Yd becomes the threshold value Ydth. Further, if a threshold value Ydth of the time Yd is set, the processor 25 may set, in addition to the upper limit value Pmax_HF or the upper limit value Imax_HF, a threshold value Ydth of the time Yd associated with the termination condition of the output of the electrical energy to differing values for the first mode and the second mode, in the same manner as the threshold value Zth_HF of the twenty-first and the twenty-third examples.

In a twenty-fourth example, the processor 25 performs the output control on the electrical energy based on the result of the determination of the heat denaturation in accordance with a pattern B7. In this case, the processor 25 sets a rate εa of decrease (setting value) with time in the output electric power P_HF immediately after the power reaches the peak value Pp_HF. Immediately after the detection of the peak value Pp_HF, the processor 25 performs the output control on the first electrical energy to decrease the output electric power P_HF at the rate εa of decrease. In this example, the processor 25 sets the rate εa of decrease, which is a variation rate with time in the output electric power P_HF, to differing values for the first mode and the second mode. This causes the processor to operate the treatment instrument 2 in states different from each other in the first and second modes. In this example, if the determination parameter η is set to the value η1, the processor 25 performs the output control to decrease the output electric power P_HF with time at a rate εa1 of decrease immediately after the detection of the peak value Pp_HF. On the other hand, when the determination parameter η is set to the value η2, the processor 25 performs the output control to decrease the output electric power P_HF with time at a rate εa2 of decrease, which is greater than the rate εa1 of decrease. Therefore, in the second mode, the processor 25 more rapidly lowers the output electric power P_HF immediately after the detection of the peak value Pp_HF, as compared with the first mode. Thus, in the second mode, the processor 25 suppresses the output of the first electrical energy from the electric power source device (generator) 3 to the treatment instrument 2 as compared with the first mode.

FIG. 22 shows an example of a variation with time in the output electric power P_HF in this example. In FIG. 22, the abscissa axis indicates time t, and the ordinate axis indicates the output electric power P_HF. In FIG. 22, the variation with time in the output electric power P_HF in the state X1 is indicated by the solid line, and the variation with time in the output electric power P_HF in the state X2 is indicated by the broken line. As shown in FIG. 22, in the state X1, immediately after the detection of the peak value Pp1_HF, the processor 25 performs the control to decrease the output electric power P_HF at the rate εa1 of decrease. On the other hand, in the state X2, immediately after the detection of the peak value Pp2_HF, the processor 25 performs the control to decrease the output electric power P_HF at the rate εa2 of decrease, which is greater than the rate εa1 of decrease. Thus, in the state X2, immediately after the detection of the peak value Pp_HF, the output electric power P_HF from the output source 31 is suppressed and the output of the first electrical energy to the electrodes 21 and 22 is suppressed as compared with the state X1. Therefore, in the state X2, the application of the high-frequency current to the treatment target is suppressed as compared with the state X1. Accordingly, also in this example, in the state X2, the appropriate treatment performance can be exhibited. This also applies to the twentieth example.

In a twenty-fifth example, the processor 25 performs the output control on the electrical energy based on the result of the determination of the heat denaturation in accordance with a pattern B8. In this example, if the determination parameter η is set to the value η1, the processor 25 causes the first electrical energy to be continuously output. On the other hand, if the determination parameter η is set to the value η2, the processor 25 causes the first electrical energy to be intermittently output. This causes the processor 25 to operate the treatment instrument 2 in states different from each other in the first mode and the second mode. Thus, in the second mode, the processor 25 suppresses the output of the first electrical energy from the electric power source device (generator) 3 to the treatment instrument 2 as compared with the first mode. In this example, the processor 25 sets the duty ratio indicating the ratio at which the first electrical energy is output in a certain period of time to different values in the first mode and the second mode. In this example, if it is determined that the treatment target has been heat-denatured prior to receiving the output command, the processor 25 switches the operation state of the treatment instrument 2 from the first mode to the second mode at the start of the third phase, for example.

FIG. 23 shows an example of variation with time in ON-OFF states of the output of the first electrical energy (HF output) in this example. In FIG. 23, the abscissa axis indicates time t, and the ordinate axis indicates ON-OFF states of the HF output. In FIG. 23, the variation with time in the ON-OFF states in the state X1 is indicated by the solid line, and the variation with time in the ON-OFF states in the state X2 is indicated by the broken line. As shown in FIG. 23, in the state X1, the processor 25 causes the first electrical energy to be continuously output even after the time t1 when the third phase is started. On the other hand, in the state X2, the processor 25 causes the first electrical energy to be intermittently output after the time t2 at which the third phase is started. Therefore, in the state X2, the output of the first electrical energy from the output source 31 to the electrodes 21 and 22 is suppressed and the application of the high-frequency current to the treatment target is suppressed, for example after the output control in the second phase is completed, compared with the state X1. Accordingly, also in this example, in the state X2, the appropriate treatment performance can be exhibited.

When the processor 25 makes an output control in accordance with any of the patterns B1 to B8, B′ 5 and B′6, the processor 25 sets any of the setting values associated with the output control on the first electrical energy to the electrodes 21 and 22 to differing values for the first mode and the second mode. Thus, in the second mode, the processor 25 suppresses the output of the first electrical energy as compared with the first mode. In one example, in the second mode, the processor 25 sets a setting value other than the setting values (Va_HF, αa, βc, Pmax_HF, Imax_HF, Zth, εa, etc.) among the setting values associated with the output control on the first electrical energy to a value different from that in the first mode. In this case, the processor 25 also suppresses the output of the first electrical energy in the second mode as compared with the first mode. The setting values associated with the output control on the first electrical energy include the setting values associated with any one of the output current I_HF, the output voltage V_HF, the output electric power P_HF, and the impedance Z_HF, the setting values relating to the variation rate with time in any one of the output current I_HF, the output voltage V_HF, the output electric power P_HF, and the impedance Z_HF, the setting values associated with the output time of the first electrical energy, and the setting values associated with the termination condition of the output of the first electrical energy. In the treatment system 1 in which the heater 23 is not provided and the electrical energy (first electrical energy) is output only to the electrodes 21 and 22 from the electric power source device 3, the processor 25 can perform the output control in accordance with any of the patterns B1 to B8, B′5, and B′6.

In a twenty-sixth example, the processor 25 performs the output control on the electrical energy based on the result of the determination of the heat denaturation in accordance with a pattern B9. In this case, the processor 25 sets the rate γb of increase in the temperature T_HT of the heater 23 until the temperature reaches the target temperature Ttar_HT. Then, for example, after the start of the second phase, the processor 25 performs the output control on the second electrical energy to increase the temperature T_HT with time at the rate γb of increase to the target temperature Ttar_HT. In this example, in the control to increase the temperature T_HT with time at the rate γb of increase, the processor 25 sets the rate γb of increase to differing values for the first mode and the second mode. That is, the processor 25 sets the rate γb of increase, which is a variation rate with time in the temperature T_HT, to differing values for the first mode and the second mode. This causes the processor 25 to operate the treatment instrument 2 in states different from each other in the first mode and the second mode. In this example, if the determination parameter η is set to the value η1, the processor 25 sets the rate (setting value) γb1 of increase. On the other hand, if the determination parameter η is set to the value η2, the processor 25 sets the rate (setting value) γb2 of increase, which is smaller than the rate γb1 of increase. Thus, in the second mode, the processor 25 suppresses the output of the second electrical energy from the electric power source device (generator) 3 to the treatment instrument 2 as compared with the first mode.

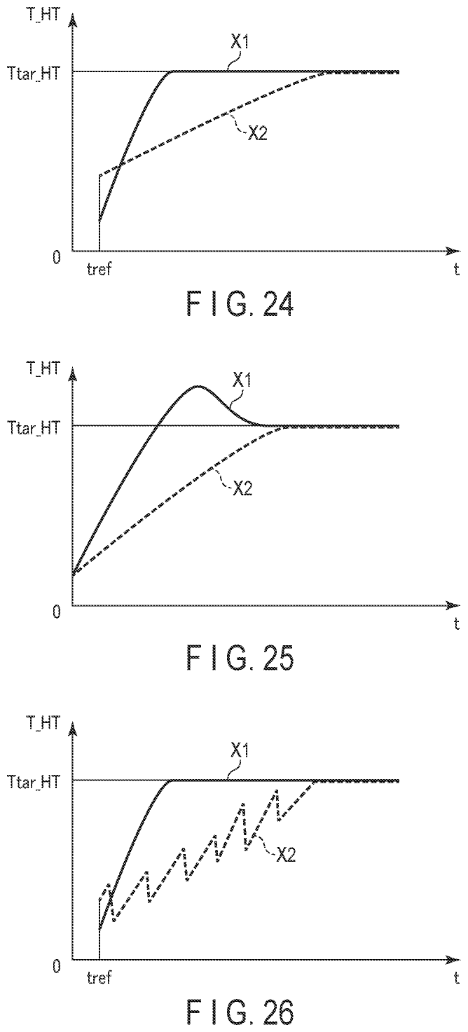

FIG. 24 shows an example of the target trajectory of the temperature T_HT in this example. In FIG. 24, the abscissa axis indicates time t, and the ordinate axis indicates the temperature T_HT. FIG. 24 shows target trajectories of the temperature T_HT after the start of the second phase, and the target trajectory of the temperature T_HT in the state X1 is indicated by the solid line, while the target trajectory of the temperature T_HT in the state X2 is indicated by the broken line. As shown in FIG. 24, in the state X1, the processor 25 performs the control to increase the temperature T_HT at the rate γb1 of increase with time until the temperature reaches the target temperature T_HT after the time tref. On the other hand, in the state X2, the processor 25 performs the control to increase the temperature T_HT at the rate γb2 of increase with time, which is smaller than the rate γb1 of increase, after the time tref. Therefore, in the state X2, the output of the second electrical energy from the output source 41 to the heater 23 is suppressed until the temperature T_HT reaches the target temperature Ttar_HT as compared with the state X1. That is, in the state X2 in which the treatment target has been heat-denatured prior to the output command, the processor 25 suppresses the output of the second electrical energy to the treatment instrument 2 as compared with the state X1 in which the treatment target has not been heat-denatured prior to the output command. Thus, in the state X2, the application of heat (treatment energy) from the heater 23 to the treatment target is suppressed as compared with the state X1. Accordingly, also in this example, in the state X2, the sealing performance and the like of the treatment target can be appropriately secured and the appropriate treatment performance can be exhibited.

In a twenty-seventh example, the processor 25 performs the output control on the electrical energy based on the result of the determination of the heat denaturation in accordance with a pattern B10. In this case, the processor 25 sets the upper limit value (setting value) Pmax_HT of an outputtable range of the output electric power P_HT to differing values for the first and second modes. This causes the processor 25 to operate the treatment instrument 2 in states different from each other in the first mode and the second mode. In this example, if the determination parameter η is set to the value η1, the processor 25 sets the upper limit value Pmax1_HT for the outputtable range of the output electric power P_HT. On the other hand, if the determination parameter η is set to the value η2, the processor 25 sets the upper limit value Pmax2_HT, which is smaller than the upper limit value Pmax1_HT, for the outputtable range of the output electric power P_HT. Thus, in the second mode, the processor 25 suppresses the output electric power P_HT from the output source 41 and suppresses the output of the second electrical energy from the electric power source device (generator) 3 to the treatment instrument 2 as compared with the first mode.

In the state X1, the processor 25 causes the output electric power P_HT to be output below the upper limit value Pmax1_HT. On the other hand, in the state X2, the processor 25 causes the output electric power P_HT to be output below the upper limit value Pmax2_HT, which is smaller than the upper limit value Pmax1_HT. Thus, in the state X2, the output of the second electrical energy to the heater 23 is suppressed as compared with the state X1. Therefore, in the state X2, the application of the heat from the heater 23 to the treatment target is suppressed as compared with the state X1. Accordingly, also in this example, in the state X2, the appropriate treatment performance can be exhibited.

In one example, the processor 25 sets either one of the upper limit value (setting value) Imax_HT of the outputtable range of the output current I_HT and the upper limit value (setting value) Vmax_HT of the outputtable range of the output voltage V_HT to differing values for the first and second modes. In this case also, in the second mode, the processor 25 suppresses the output of the second electrical energy to the treatment instrument 2 as compared with the first mode. Therefore, in the state X2, the application of the heat from the heater 23 to the treatment target is suppressed as compared with the state X1. Accordingly, also in these examples, in the state X2, the appropriate treatment performance can be exhibited.

In a twenty-eighth example, the processor 25 performs the output control on the electrical energy based on the result of the determination of the heat denaturation in accordance with a pattern B11. In this case, the processor 25 sets the initial value Pe_HT of the output electric power P_HT. Then, the processor 25 causes the second electrical energy at the initial value Pe_HT to be output to the heater 23 at and immediately after the output start time. In this example, the processor 25 sets the initial value Pe_HT to differing values for the first mode and the second mode. This causes the processor 25 to operate the treatment instrument 2 in states different from each other in the first mode and the second mode. In this example, if the determination parameter η is set to the value η1, the processor 25 sets an initial value (setting value) Pe1_HT. On the other hand, if the determination parameter η is set to the value η2, the processor 25 sets an initial value (setting value) Pe2_HT, which is smaller than the initial value Pe1_HT. Thus, in the second mode, the processor 25 suppresses the output of the second electrical energy from the electric power source device (generator) 3 to the treatment instrument 2 as compared with the first mode. In this example, the processor 25 sets the determination parameter η prior to the output start time (t=0). If it is determined that the treatment target has been heat-denatured prior to receiving the output command, the processor 25 causes the treatment instrument 2 to operate in the second mode at the output start time. It should be noted that, by making a determination, for example in accordance with any of the patterns A12 to A15 described above, the processor 25 can determine the heat denaturation of the treatment target even before the output start time.

FIG. 25 shows an example of a variation with time in the temperature T_HT in this example. In FIG. 25, the abscissa axis indicates time t, and the ordinate axis indicates the temperature T_HT. In FIG. 25, the variation with time in the temperature T_HT in the state X1 is indicated by the solid line, and the variation with time in the temperature T_HT in the state X2 is indicated by the broken line. In the state X1, the processor 25 in this example causes the second electrical energy at the initial value Pe1_HT to be output at and immediately after the output start time. On the other hand, in the state X2, the processor 25 causes the second electrical energy at the initial value Pe2_HT, which is smaller than the initial value Pe1_HT, to be output at and immediately after the output start time. Thus, in the state X2, the output of the second electrical energy to the heater 23 is suppressed, and the application of the heat from the heater 23 to the treatment target is suppressed as compared with the state X1. Further, as shown in FIG. 25, in the state X2, the temperature T_HT more gradually increases to the target temperature Ttar_HT as compared with the state X1. In the state X2, the overshoot of the temperature T_HT to a temperature higher than the target temperature Ttar_HT is suppressed. Accordingly, also in this example, in the state X2, the appropriate treatment performance can be exhibited.

In one example, the processor 25 sets either one of the initial value (setting value) Ie_HT of the output current I_HT and the initial value (setting value) Ve_HT of the output voltage V_HT to differing values for the first and second modes. In this case also, in the second mode, the processor 25 suppresses the output of the second electrical energy to the treatment instrument 2 as compared with the first mode. Therefore, in the state X2, the application of the heat from the heater 23 to the treatment target is suppressed as compared with the state X1. Accordingly, also in these examples, in the state X2, the appropriate treatment performance can be exhibited.

In a twenty-ninth example, the processor 25 performs the output control on the electrical energy based on the result of the determination of the heat denaturation in accordance with a pattern B12. Also in this case, the processor 25 causes the second electrical energy at the initial value Pe_HT to be output to the heater 23 at and immediately after the output start time. In this example, the processor 25 sets a time Ye for outputting the second electrical energy at the initial value Pe_HT from the output start time (t=0) to differing values for the first mode and the second mode. This causes the processor 25 to operate the treatment instrument 2 in states different from each other in the first mode and the second mode. In this example, if the determination parameter η is set to the value η1, the processor 25 sets a time (setting value) Ye1. On the other hand, if the determination parameter η is set to the value η2, the processor 25 sets a time (setting value) Ye2, which is longer than the time Ye1. Thus, in the second mode, the time Ye in which the second electrical energy is output at the initial value Pe is increased, and the processor 25 suppresses the output of the second electrical energy from the electric power source device (generator) 3 to the treatment instrument 2 as compared with the first mode. Thus, in this example, before or immediately after the output start time (t=0), the processor 25 determines whether or not the treatment target has been heat-denatured prior to receiving the output command.

In the state X1, the processor 25 causes the second electrical energy to be output at the initial value Pe_HT during a period of the time Ye1 from the output start time. On the other hand, in the state X2, the processor 25 causes the second electrical energy to be output at the initial value Pe_HT during a period of the time Ye2, which is longer than the time Ye1, from the output start time. Thus, in the state X2, the output of the second electrical energy from the output source 41 to the heater 23 is suppressed, and the application of the heat from the heater 23 to the treatment target is suppressed as compared with the state X1. In this example, the temperature T_HT of the heater 23 varies with time in each of the states X1 and X2 as in the twenty-eighth example. Accordingly, also in this example, in the state X2, the appropriate treatment performance can be exhibited.

In one example, the processor 25 sets either of the output time at the initial value Ie_HT from the output start time and the output time at the initial value Ve_HT from the output start time to differing values for the first mode and the second mode. In this case also, in the second mode, the processor 25 suppresses the output of the second electrical energy to the treatment instrument 2 as compared with the first mode. Therefore, in the state X2, the application of the heat from the heater 23 to the treatment target is suppressed as compared with the state X1. Accordingly, also in these examples, in the state X2, the appropriate treatment performance can be exhibited.

In a thirtieth example, the processor 25 performs the output control on the electrical energy based on the result of the determination of the heat denaturation in accordance with a pattern B13. In this case, the processor 25 performs PD control or PID control at the target temperature Ttar_HT for the output of the second electrical energy. The processor 25 sets a differential gain Kd of the differential term of the PD control or the PID control to differing values for the first mode and the second mode. That is, in the second mode, the processor 25 sets the differential gain Kd, which is the setting value associated with the term of the differential operation, to a value different from that of the first mode. This causes the processor 25 to operate the treatment instrument 2 in states different from each other in the first mode and the second mode. In this example, if the determination parameter η is set to the value η1, the processor 25 sets a differential gain (setting value) Kd1. On the other hand, if the determination parameter η is set to the value η2, the processor 25 sets a differential gain (setting value) Kd2, which is greater than the differential gain Kd1. Thus, in the second mode, the processor 25 suppresses the output of the second electrical energy from the electric power source device (generator) 3 to the treatment instrument 2 as compared with the first mode.

The processor 25 performs PD control or PID control with the differential gain Kd1 in the state X1. On the other hand, in the state X2, the processor 25 performs PD control or PID control with the differential gain Kd2 different from the differential gain Kd1. The differential gains Kd are set to different values in the states X1 and X2; therefore, in the state X2, the output of the second electrical energy from the output source 41 to the heater 23 is suppressed, and the application of the heat from the heater 23 to the treatment target is suppressed as compared with the state X1. In this example, the temperature T_HT of the heater 23 varies with time in each of the states X1 and X2 as in the twenty-eighth example and the twenty-ninth example. Accordingly, also in this example, in the state X2, the appropriate treatment performance can be exhibited.

In a thirty-first example, the processor 25 performs the output control on the electrical energy based on the result of the determination of the heat denaturation in accordance with a pattern B14. In this case, the processor 25 sets a target trajectory of the temperature T_HT of the heater 23. The processor 25 performs the output control on the second electrical energy to change the temperature T_HT along the set target trajectory, for example after the start of the second phase. In this example, the processor 25 sets target trajectories different from each other for the first mode and the second mode. This causes the processor 25 to operate the treatment instrument 2 in states different from each other in the first and second modes. In this example, if the determination parameter η is set to the value η1, the processor 25 sets a first target trajectory of the temperature T_HT. On the other hand, if the determination parameter η is set to the value η2, the processor 25 sets a second target trajectory of the temperature T_HT. In the control along the second target trajectory, the processor 25 suppresses the output of the second electrical energy from the electric power source device (generator) 3 to the treatment instrument 2 as compared with the control along the first target trajectory.

FIG. 26 shows an example of the target trajectory of the temperature T_HT in this example. In FIG. 26, the abscissa axis indicates time t, and the ordinate axis indicates the temperature T_HT. FIG. 26 shows target trajectories of the temperature T_HT after the start of the second phase, and the target trajectory of the temperature T_HT in the state X1 is indicated by the solid line, while the target trajectory of the temperature T_HT in the state X2 is indicated by the broken line. As shown in FIG. 26, the processor 25 sets the first target trajectory of the temperature T_HT in the state X1. In the first target trajectory, the temperature T_HT linearly increases to the target temperature Ttar_HT. The processor 25 sets the second target trajectory of the temperature T_HT in the state X2. In the second target trajectory, the temperature T_HT increases in stages to the target temperature Ttar_HT, while repeatedly increasing and decreasing. In the case of changing the temperature T_HT along the second target trajectory, the average value of the rate of increase in the temperature T_HT up to the target temperature Ttar_HT is smaller than in the case of changing the temperature T_HT along the first target trajectory. Thus, in the state X2, the output of the second electrical energy from the output source 41 to the heater 23 is suppressed, and the application of the heat from the heater 23 to the treatment target is suppressed as compared with the state X1. Accordingly, also in this example, in the state X2, the appropriate treatment performance can be exhibited.

When the processor 25 performs the output control in accordance with any one of the patterns B9 to B14, the processor 25 sets any one of the setting values associated with the output control on the second electrical energy to the heater 23 to differing values for the first mode and the second mode. In the second mode, the processor 25 suppresses the output of the second electrical energy as compared with the first mode. In one example, in the second mode, the processor 25 sets a setting value other than the setting value (γb, Pmax_HT, Pe_Ht, Ye, Kd, etc.) described above among the setting values associated with the output control on the second electrical energy to a value different from that in the first mode. In this case, in the second mode, the processor 25 also suppresses the output of the second electrical energy as compared with the first mode. The setting values associated with the output control on the second electrical energy include the setting value for any one of the output current I_HT, the output voltage V_HT, the output electric power P_HT, and the temperature T_HT, the setting value for a variation rate with time in the temperature T_HT, the setting value for the initial value at the output start time of the second electrical energy, the setting value for a time when the second electrical energy is output at the initial value, and the setting value associated with a term of the differential operation in each of the PD control and the PID control. In the treatment system 1 in which the electrodes 21 and 22 are not provided and the electrical energy (second electrical energy) is output only to the heater 23 from the electric power source device 3, the processor 25 can also perform the output control in accordance with any of the patterns B9 to B14.

In a thirty-second example, the processor 25 performs the output control on the electrical energy based on the result of the determination of the heat denaturation in accordance with a pattern B15. Therefore, the processor 25 starts an output of only the first electrical energy based on the reception of the output command. After starting the output of the first electrical energy, the processor 25 starts an output of the second electrical energy. For example, in the first phase and the second phase, the processor 25 causes only the first electrical energy to be output, and ceases the output of the second electrical energy. The processor 25 then starts the output of the second electrical energy at any time after the start of the third phase. In this example, the processor 25 sets the time Yd from the start of the third phase to the start of the output of the second electrical energy to values different from each other for the first mode and the second mode. That is, the processor 25 sets the time Yd, which is the setting value associated with the output start time of the second electrical energy, to differing values for the first mode and the second mode. This causes the processor 25 to operate the treatment instrument 2 in states different from each other in the first mode and the second mode. In this example, if the determination parameter η is set to the value η1, the processor 25 sets a time (setting value) Yd1. The time Yd1 is, for example, 0, and in this case, the output of the second electrical energy is started from the start of the third phase. On the other hand, if the determination parameter η is set to the value η2, the processor 25 sets a time (setting value) Yd2, which is longer than the time Yd1. Thus, in the second mode, the processor 25 suppresses the output of the second electrical energy from the electric power source device (generator) 3 to the treatment instrument 2 as compared with the first mode.

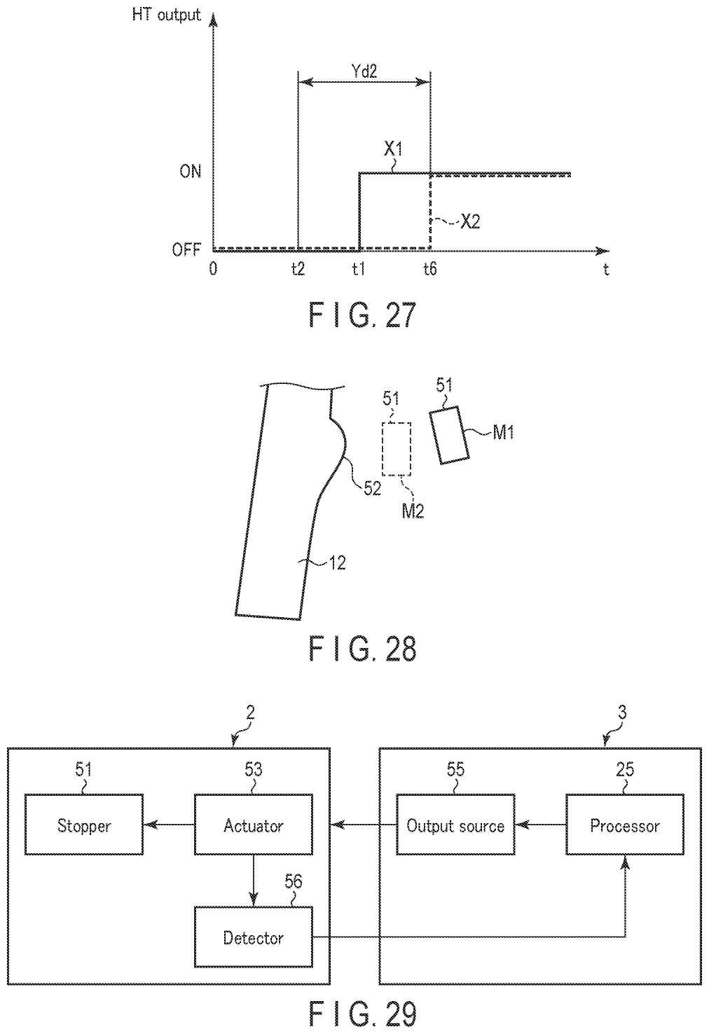

FIG. 27 shows an example of variation with time in ON-OFF states of the output of the second electrical energy (HT output) in this example. In FIG. 27, the abscissa axis indicates time t, and the ordinate axis indicates ON-OFF states of the HT output. In FIG. 27, the variation with time in the ON-OFF states in the state X1 is indicated by the solid line, and the variation with time in the ON-OFF states in the state X2 is indicated by the broken line. As shown in FIG. 27, in the state X1, the processor 25 starts an output of the second electrical energy at the time t1 when the third phase is started. On the other hand, in the state X2, the processor 25 starts the output of the second electrical energy at a time t6 when the time Yd2 has elapsed since the start of the third phase (t2). Thus, in the state X2, in the third phase, the output of the second electrical energy from the output source 41 to the heater 23 is suppressed, and the application of the heat from the heater 23 to the treatment target is suppressed as compared with the state X1. Accordingly, also in this example, in the state X2, the appropriate treatment performance can be exhibited.

In one example, as in the thirty-second example, the processor 25 starts an output of the first electrical energy based on the reception of the output command, and thereafter starts an output of the second electrical energy. However, in this example, in the second mode, the processor 25 sets a setting value other than the time Yd among the setting values associated with the output start time of the second electrical energy to a value different from that in the first mode. In this case, the processor 25 also suppresses the output of the second electrical energy in the second mode as compared with the first mode.

In a thirty-third example, the processor 25 performs the output control on the electrical energy based on the result of the determination of the heat denaturation in accordance with a pattern B16. In this example, if the determination parameter η is set to the value η1, the processor 25 causes both the first electrical energy and the second electrical energy to be output. In this example, if the determination parameter η is set to the value η2, the processor 25 causes only one of the first electrical energy and the second electrical energy to be output. This causes the processor 25 to operate the treatment instrument 2 in states different from each other in the first mode and the second mode. In the second mode, the processor 25 suppresses the output of one of the first electrical energy and the second electrical energy to the treatment instrument 2 as compared with the first mode. In the state X1, even after setting the determination parameter η, the processor 25 causes both the first electrical energy and the second electrical energy to be output. On the other hand, in the state X2, after setting the determination parameter η, the processor 25 causes only one of the first electrical energy and the second electrical energy to be output. Therefore, in the state X2, after setting the determination parameter η, one of the output of the first electrical energy and the output of the second electrical energy is suppressed, and the application of one of the high-frequency current and the heat of the heater 23 to the treatment target is suppressed, in comparison with the state X1. Accordingly, also in this example, in the state X2, the appropriate treatment performance can be exhibited.

In a thirty-fourth example, the processor 25 performs the output control on the electrical energy based on the result of the determination of the heat denaturation in accordance with a pattern B17. In this example, the processor 25 sets the setting values associated with the output control on the first electrical energy and the setting values associated with the output control on the second electrical energy to be identical to each other for the first mode and the second mode. However, in this example, the processor 25 adjusts a grasping force Fb generated between the grasping pieces 15 and 16 by output controlling on electrical energy other than the first electrical energy and the second electrical energy to the treatment instrument 2. If the determination parameter η is set to the value η1, the processor 25 causes the treatment target to be grasped by a grasping force Fb1. On the other hand, if the determination parameter η is set to the value η2, the processor 25 causes the treatment target to be grasped by a grasping force Fb2, which is smaller than the grasping force Fb1. This causes the processor 25 to operate the treatment instrument 2 in states different from each other in the first mode and the second mode.

In the state X1, after setting the determination parameter η, the processor 25 causes the treatment target to be grasped by the grasping force Fb1. On the other hand, in the state X2, after setting the determination parameter η, the processor 25 causes the treatment target to be grasped by the grasping force Fb2, which is smaller than the grasping force Fb1. Therefore, in the state X2, after setting the determination parameter η, the grasping force Fb for grasping the treatment target is smaller than in the state X1. That is, in this example, in the state X2 in which the treatment target has been heat-denatured prior to the output command, the processor 25 decreases the grasping force Fb for grasping the treatment target as compared with the state X1 in which the treatment target has not been heat-denatured prior to the output command. Therefore, in the state X2, the pressure that acts on the treatment target is suppressed as compared with the state X1. Accordingly, also in this example, in the state X2 in which the treatment energy is applied twice or more times to the already heat-denatured portion, the appropriate treatment performance can be exhibited.

FIG. 28 shows an example of a configuration to change the grasping force Fb at the end effector 7. In the configuration of FIG. 28, a stopper 51 is provided inside the housing 6. The handle 12 is closed with respect to the grip 11 until the contact 52 abuts against the stopper 51. The stopper 51 is movable between a position M1 in the first mode and a position M2 in the second mode. Therefore, in the second mode, the stroke in the closing operation of the handle 12 is smaller and the grasping force Fb is decreased as compared with the first mode.

FIG. 29 shows a configuration to move the stopper 51. In this example, the treatment instrument 2 includes an actuator 53 configured to move the stopper 51, and the electric power source device 3 includes an output source 55 different from the output sources 31 and 41. The actuator 53 is, for example, an electric motor. The output source (operating electric power supply) 55 includes a conversion circuit and a transformer, and forms a drive circuit. The output source 55 converts electric power from a battery electric power source, a commercial electric power source or the like into operating electric power (electrical energy) for the actuator 53, and outputs the operating electric power to the actuator 53. When the operating electric power is supplied to the actuator 53, the actuator is actuated to move the stopper 51. The treatment instrument 2 also includes a detector 56, such as an encoder, configured to detect the operating state of the actuator 53. The processor 25 controls the operating electric power output to the actuator 53 and controls the operation of the actuator 53 based on the result of the detection by the detector 56. The processor 25 adjusts the position of the stopper 51 and the grasping force Fb by controlling the operation of the actuator 53.

In one example, the handle 12 is connected to the movable member 13 via an elastic member (not shown). The processor 25 controls the amount of contraction of the elastic member by controlling the operation of the actuator (for example, 53). Thus, the processor 25 adjusts the elastic force from the elastic member to the movable member 13 to adjust the grasping force Fb.

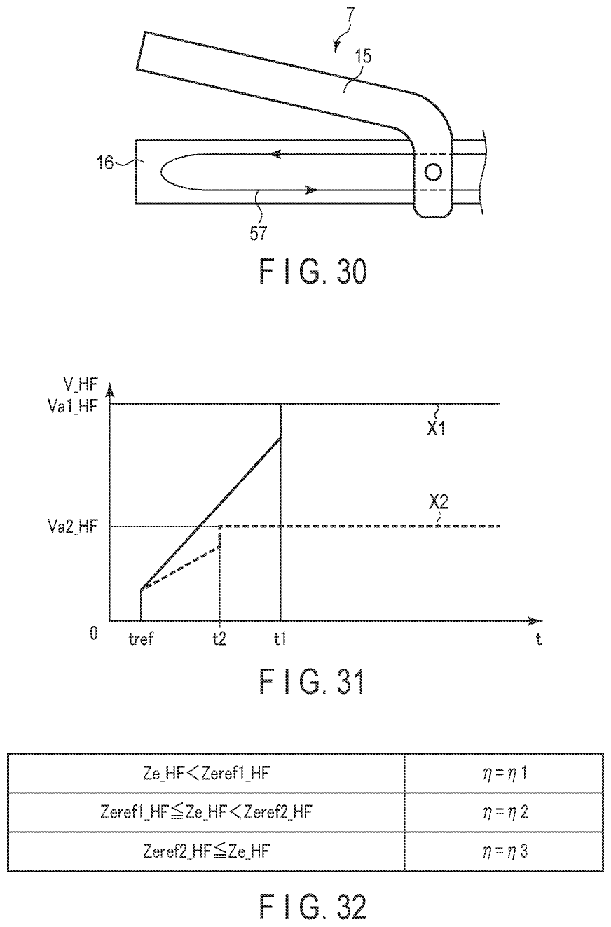

In a thirty-fifth example, the processor 25 performs the output control on the electrical energy based on the result of the determination of the heat denaturation in accordance with a pattern B18. In this example, the processor 25 also sets the setting values associated with the output control of the first electrical energy and the setting values associated with the output control of the second electrical energy to be identical to each other for the first mode and the second mode. In this embodiment, as shown in FIG. 30, a flow path 57 is formed in the grasping piece 16. The treatment instrument 2 includes an actuator 53 such as a pump, and the actuator 53 is actuated to cause cooling water to flow into the flow path 57. In this example, the processor 25 also controls the output of the operating electric power to the actuator 53. As a result, the processor 25 controls the operation of the actuator 53 to adjust whether or not to cause the cooling water to flow into the flow path 57. In the present example, if the determination parameter η is set to the value η1, the processor 25 ceases the operation of the actuator 53 and does not cause the cooling water to flow into the flow path 57. On the other hand, if the determination parameter η is set to the value η2, the processor 25 causes the actuator 53 to operate and causes the cooling water to flow into the flow path 57. This causes the processor 25 to operate the treatment instrument 2 in differing states for the first mode and the second mode.

In the state X1, even after setting the determination parameter η, the processor 25 does not cause the cooling water to flow into the flow path 57. On the other hand, in the processor 25, in the state X2, after setting the determination parameter η, the processor 25 causes the cooling water to flow into the flow path 57. That is, in the present example, in the state X2 in which the treatment target has been heat-denatured prior to the output command, the cooling water is caused to flow into the flow path 57 of the end effector 7 under the control of the processor 25. Then, in the state in which the end effector 7 is cooled by the cooling water, the treatment energy is applied to the treatment target. Accordingly, in the state X2, the appropriate treatment performance can be exhibited.

In one example, the processor 25 sets a plurality of values out of the setting values mentioned above to differing values for the first mode and the second mode. For example, in a thirty-sixth example, the processor 25 sets the target value Va_HF of the output voltage V_HF in the third phase and the rate αa of increase in the output voltage V_HF in the second phase to differing values for the first mode and the second mode. In this example, the processor 25 sets the target value Va_HF in accordance with the pattern B1, and sets the rate αa of increase in accordance with the pattern B2.

FIG. 31 shows an example of the target trajectory of the output voltage V_HF in this example. In FIG. 31, the abscissa axis indicates time t, and the ordinate axis indicates the output voltage V_HF. FIG. 31 shows target trajectories of the output voltage V_HF in the second phase and the third phase, and the target trajectory of the output voltage V_HF in the state X1 is indicated by the solid line, while the target trajectory of the output voltage V_HF in the state X2 is indicated by the broken line. As shown in FIG. 31, the processor 25 performs the control to increase the output voltage V_HF at the rate αa1 of increase in the second phase in the state X1. Then, the processor 25 performs the constant voltage control on the output voltage V_HF at the target value Va1_HF from the start of the third phase (t=t1). On the other hand, in the state X2, the processor 25 performs the control to increase the output voltage V_HF at the rate αa2 of increase, which is smaller than the rate αa1 of increase, in the second phase. Then, the processor 25 performs the constant voltage control on the output voltage V_HF at the target value Va2_HF, which is smaller than the target value Va1_HF from the start of the third phase (t=t2). In this example also, in the state X2 in which the treatment target has been heat-denatured prior to the output command, the output of the first electrical energy to the treatment instrument 2 is suppressed and the application of the high-frequency current to the treatment target is suppressed, as compared with the state X1 in which the treatment target has not been heat-denatured prior to the output command.

Accordingly, also in this example, in the state X2, the appropriate treatment performance can be exhibited.

[Combination of Determination of Heat Denaturation of Treatment Target and Output Control on Electrical Energy Based on Determination Result]

The determination of the heat denaturation of the treatment target and the output control on the electrical energy based on the determination result of the heat denaturation can be appropriately combined, insofar as they do not contradict. For example, any one or more of the determinations of the patterns A1 to A15 shown in FIG. 8 and any one or more of the output controls of the patterns B1 to B18, B′5, and B′6 shown in FIG. 17 can be appropriately combined, insofar as they do not contradict.

(Modification)

In one modification, the processor 25 causes the treatment instrument 2 to operate in a determination mode, which is different from the first and second modes, at the output start time of the electrical energy to the treatment instrument 2. In this case, in the determination mode, the processor 25 sets, for example, any one of the setting values associated with the output control on the first electrical energy and the setting values associated with the output control on the second electrical energy to differing values for the first mode and the second mode. In this modification, if it is determined that the treatment target has not been heat-denatured prior to receiving the output command, that is, if the determination parameter η is set to the value η1, the processor 25 switches the operation state of the treatment instrument 2 from the determination mode to the first mode. On the other hand, if it is determined that the treatment target has been heat-denatured prior to receiving the output command, that is, if the determination parameter η is set to the value η2, the processor 25 switches the operation state of the treatment instrument 2 from the determination mode to the second mode.

In one modification, the processor 25 selects an operation state of the treatment instrument 2 from three or more modes based on the result of determination of the heat denaturation of the treatment target. In a first modification, the processor 25 selects an operation state of the treatment instrument 2 from three modes based on the result of determination of the heat denaturation of the treatment target. In this case, the processor 25 sets the determination parameter η to any one of the values η1 to η3. Also, in this modification, the processor 25 determines whether or not the treatment target has been heat-denatured prior to receiving the output command based on any one of the parameters described above, and sets a determination parameter η. If the determination parameter η is set to the value η1, the processor 25 determines that the treatment target has not been heat-denatured, and selects the first mode as the operation state of the treatment instrument 2. If the determination parameter η is set to the value η2, the processor 25 determines that the treatment energy has been applied to the treatment target only once prior to the output command, and selects the second mode as the operation state of the treatment instrument 2. If the determination parameter η is set to the value η3, the processor 25 determines that the treatment energy has been applied to the treatment target twice or more times prior to the output command, and selects the third mode as the operation state of the treatment instrument 3.

FIG. 32 shows an example of determination of heat denaturation of the treatment target by the processor 25 of this modification. In an example of FIG. 32, the processor 25 determines the heat denaturation of the treatment target based on an initial value Ze_HF of an impedance Z_HF. In this example, if the initial value Ze_HF is smaller than a first reference value Zeref1_HF, the processor 25 sets the determination parameter η to the value η1 and causes the treatment instrument 2 to operate in the first mode. If the initial value Ze_HF is equal to or greater than the first reference value Zeref1_HF and smaller than a second reference value Zeref2_HF, the processor 25 sets the determination parameter η to the value η2 and causes the treatment instrument 2 to operate in the second mode. Then, if the initial value Ze_HF is equal to or greater than the second reference value Zeref2_HF, the processor 25 sets the determination parameter η to the value η3 and causes the treatment instrument 2 to operate in the third mode. The processor 25 sets the second reference value Zeref2_HF to be greater than the first reference value Zeref1_HF.

In this modification, the processor sets any one of the setting values associated with the output control on the first electrical energy and the setting values associated with the output control on the second electrical energy to values different from one another for the first mode, the second mode, and the third mode. Accordingly, in the second mode, the processor 25 suppresses the output of at least one of the first electrical energy or the second electrical energy as compared with the first mode. Furthermore, in the third mode, the processor 25 suppresses the output of at least one of the first electrical energy or the second electrical energy as compared with the second mode.

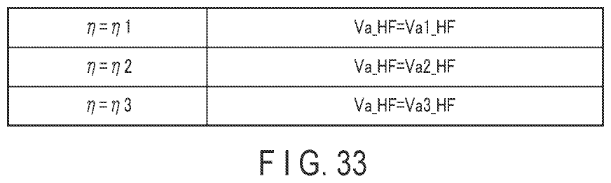

FIG. 33 shows an example of setting values of the first mode, the second mode, and the third mode set by the processor 25 of the present modification. In the example of FIG. 33, the processor 25 sets the target value Va_HF of the output voltage V_HF to values different from one another for the first mode, the second mode, and the third mode. In this example, if the determination parameter η is set to the value η1, that is, if the first mode is selected, the processor 25 performs the constant voltage control at the target value Va1_HF. If the determination parameter η is set to the value η2, that is, if the second mode is selected, the processor 25 performs the constant voltage control at the target value Va2_HF, which is smaller than the target value Va1_HF. If the determination parameter η is set to the value η3, that is, if the third mode is selected, the processor 25 performs the constant voltage control at the target value Va3_HF, which is smaller than the target value Va2_HF.

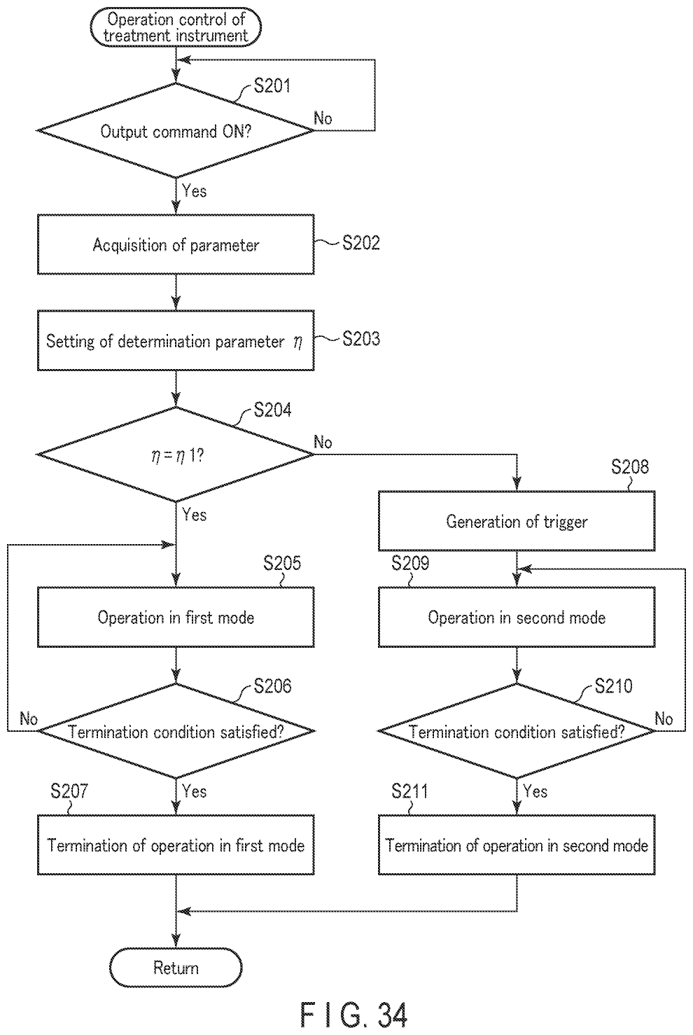

In a second modification, the processor 25 determines heat denaturation of the treatment target before starting an output of electrical energy to the treatment instrument 2, for example, before starting the output control in the first phase described above. For example, by performing the determination in accordance with any one of the patterns A12 to A15 described above, the processor 25 can determine the heat denaturation of the treatment target even before the output start time. FIG. 34 is a flowchart showing the processing performed by the processor 25 in an operation control of the treatment instrument 2 of the present modification. As shown in FIG. 34, in the present modification, when the processor 25 receives the output command (S201—Yes), the processor 25 acquires the parameters described above for use in the determination of the heat denaturation of the treatment target (S202). The processor 25 sets a determination parameter η based on the acquired parameter (S203). When the determination parameter η is set, the processing proceeds to S204. In this modification, after the processing of S203 is performed, the processor 25 starts the output of electrical energy to the treatment instrument 2 and activates the treatment instrument 2.

If the determination parameter η is set to the value η1 (S204—Yes), the processor 25 starts the output of the electrical energy to the treatment instrument 2 and causes the treatment instrument 2 to operate in the first mode (S205). If the termination condition is not satisfied (S206—No), the processor 25 continuously causes the treatment instrument 2 to operate in the first mode. If the termination condition is satisfied (S206—Yes), the processor 25 terminates the operation in the first mode of the treatment instrument 2 (S207). On the other hand, if the determination parameter η is set to the value η2 (S204—No), the processor 25 generates a trigger (S208). The processor 25 then starts an output of the electrical energy to the treatment instrument 2 and causes the treatment instrument 2 to operate in the second mode (S209). If the termination condition is not satisfied (S210—No), the processor 25 continuously causes the treatment instrument 2 to operate in the second mode. If the termination condition is satisfied (S210—Yes), the processor 25 terminates the operation in the second mode of the treatment instrument 2 (S211).

In one modification, the processor 25 selects an operation state of the treatment instrument 2 based on an operation by the operator with the touch screen 27 or the like. In this case, the operation state of the treatment target is switched between the first mode and the second mode by the operation of the operator. In this modification, before or immediately after the start of the output of the electrical energy, the operator determines whether or not the treatment target has already been heat-denatured before the present output, for example, whether the treatment target is in either the state X1 or X2. On the basis of the determination result, the operator sets the operation state of the treatment instrument in the touch screen 27 or the like. The processor then selects the set operation states and causes the treatment instrument 2 to operate at the selected operation state (for example, the one selected from the first mode and the second mode).

Furthermore, in one modification, a notification member (not shown) is provided in the electric power source device 3 or separately from the electric power source device 3. In this case, if the processor 25 sets the determination parameter η to the value η2 and generates the trigger, the processor 25 activates the notification member. In this modification, the operator determines whether or not the treatment target has already been heat-denatured prior to the present output based on the activation state of the notification member. If the notifying member is activated, the operator determines that the treatment target has already been heat-denatured before the present output. The notification member may be, for example, a lamp, a buzzer, a display, or the like. When the notifying member is activated, light is emitted from the lamp, a buzzer sound is generated, or a screen is displayed.

In the above-described embodiment and the like, only one electric power source device 3 is provided; however, in one modification, the electric power source device to output the first electrical energy and the electric power source device to output the second electrical energy are separate devices. In this case, the electric power source device to output the first electrical energy includes the output source 31, the current detection circuit 35, the voltage detection circuit 36, and the A/D converter 37. The electric power source device to output the second electrical energy includes the output source 41, the current detection circuit 45, the voltage detection circuit 46, and the A/D converter 47. In addition, each of the electric power source devices includes a storage medium and one or more processors. The one or more processors provided in each of the electric power source devices form a control device to control the treatment system 1, and the aforementioned processing is performed.

In another modification, the treatment instrument 2 includes one or more processors that perform the processes described above, and the one or more processors provided in the treatment instrument 2 form a control device to control the treatment system 1.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A control device of a generator, the generator being configured to output electrical energy to a treatment instrument, thereby applying treatment energy that heat-denatures a treatment target from the treatment instrument to the treatment target, the control device comprising a processor configured to:
cause the generator to output the electrical energy to the treatment instrument when an output command is received by the processor;
after starting an output of the electrical energy to the treatment instrument, determine whether the treatment target was heat-denatured prior to receiving the output command;
based on determination of a heat denaturation of the treatment target, select an operation state of the treatment instrument, the operation state being one of:
a first mode when the treatment target is not heat-denatured, and in the first mode, the treatment target is sealed by the treatment energy; and
a second mode when the treatment target is heat-denatured, and in the second mode, the treatment target is sealed by the treatment energy; and
operate the treatment instrument at the selected operation state by controlling the output of the electrical energy to the treatment instrument.

2. The control device of claim 1, wherein in the second mode, the processor is configured to suppress the output of the electrical energy from the generator to the treatment instrument.

3. The control device of claim 2, wherein:
the processor is configured to:
set electrical energy setting values associated with an output control on the electrical energy to bipolar electrodes provided on the treatment instrument; and
cause the generator to output the electrical energy to the bipolar electrodes to cause a high-frequency current to flow as the treatment energy through the treatment target between the bipolar electrodes; and
in the second mode, the processor is configured to set at least one of the electrical energy setting values to a value different from that of the first mode to suppress the output of the electrical energy to the bipolar electrodes.

4. The control device of claim 3, wherein:
in the second mode, the processor is configured to set, to a value different from that of the first mode, at least one of:
a setting value relating to one of an output current, an output voltage, and output electric power to the bipolar electrodes;
a setting value relating to a variation rate with time in one of the output current, the output voltage, and the output electric power;
a setting value relating to an impedance in an electrical path of the electrical energy to the bipolar electrodes;
a setting value relating to a variation rate with time in the impedance;
a setting value associated with an output time of the electrical energy to the bipolar electrodes; or
a setting value associated with a termination condition of the output of the electrical energy to the bipolar electrodes, thereby suppressing the output of the electrical energy to the bipolar electrodes as compared with the first mode.

5. The control device of claim 2, wherein:
the processor is configured to:
set heater setting values associated with an output control on the electrical energy to a heater provided on the treatment instrument; and
cause the generator to output the electrical energy to apply heat generated by the heater to the treatment target as the treatment energy; and
in the second mode, the processor is configured to set at least one of the heater setting values to a value different from that of the first mode to suppress the output of the electrical energy to the heater in the second mode.

6. The control device of claim 5, wherein in the second mode, the processor is configured to set, a value different from that of the first mode, for at least one of:
a setting value relating to one of an output current, an output voltage, and output electric power to the heater;
a setting value relating to a temperature of the heater;
a setting value relating to a variation rate with time in the temperature of the heater;
an initial value of the electrical energy to the heater at an output start time;
a setting value for a time when the electrical energy is output to the heater at the initial value; or
a setting value associated with a term of a differential operation in each of PD control and PID control on the temperature of the heater to suppress the output of the electrical energy to the heater.

7. The control device of claim 2, wherein: the processor is configured to:
cause the generator to output a first electrical energy to bipolar electrodes provided in the treatment instrument to cause a high-frequency current to flow as the treatment energy through the treatment target between the bipolar electrodes, and
cause the generator to output a second electrical energy to a heater provided in the treatment instrument to cause heat generated by the heater to be applied as the treatment energy to the treatment target, and
in the second mode, the processor is configured to set a value different from that of the first mode, for at least one of:
a setting value associated with an output control of the first electrical energy to the bipolar electrodes; or
a setting value associated with an output control of the second electrical energy to the heater to suppress the output of the electrical energy to the treatment instrument.

8. The control device of claim 7, wherein:
the processor is configured to:
set a time setting value associated with an output start time of the second electrical energy to the treatment instrument; and
initiate an output of the first electrical energy based on the output command received by the processor, and
subsequently initiate an output of the second electrical energy; and
in the second mode, the processor is configured to set the time setting value to a value different from that of the first mode to suppress the output of the electrical energy to the treatment instrument.

9. The control device of claim 7, wherein:
the processor is configured to:
output both the first electrical energy and the second electrical energy in the first mode; and
output only one of the first electrical energy or the second electrical energy in the second mode to suppress the output of the electrical energy to the treatment instrument.

10. The control device of claim 1, wherein the processor is configured to:
acquire a parameter indicative of a reaction of the treatment target to an application of the treatment energy, in a state in which the treatment energy from the treatment instrument is applied to the treatment target from a supply of the electrical energy to the treatment instrument; and
determine, based on the acquired parameter, the heat denaturation of the treatment target.

11. The control device of claim 1, wherein:
the processor is configured to operate the treatment instrument in the first mode at an output start time of the electrical energy, and
if the processor determines that the treatment target has been heat-denatured, the processor is configured to switch the operation state of the treatment instrument from the first mode to the second mode.

12. A treatment system comprising:
a treatment instrument including an end effector configured to apply treatment energy that heat-denatures a treatment target;
a generator configured to output electrical energy to the treatment instrument; and
an endoscope system configured to acquire an image of the treatment target,
the generator including a processor configured to:
cause the generator to output the electrical energy to the treatment instrument based on an output command received by the processor;
acquire from the endoscope system a parameter indicative of a reaction of the treatment target to an application of the treatment energy after starting an output of the electrical energy to the treatment instrument;
determine, based on the acquired parameter, whether the treatment target has been heat-denatured prior to receiving the output command; and
select an operation mode of the treatment instrument based on a result of the determination of whether the treatment target is heat-denatured.

13. An operation method of a control device of a generator, the generator being configured to output electrical energy to a treatment instrument, thereby applying treatment energy that heat-denatures a treatment target from the treatment instrument to the treatment target, the method comprising:
outputting the electrical energy from the generator to the treatment instrument based on an output command received by a processor;
after starting an output of the electrical energy to the treatment instrument, determining whether the treatment target was heat-denatured prior to receiving the output command;
based on the determination of whether the treatment target was heat-denatured, selecting, as an operation state of the treatment instrument, one of:
a first mode when the treatment target is not heat-denatured; and
a second mode when the treatment target is heat-denatured; and
operating the treatment instrument at a selected operation state by controlling the output of the electrical energy to the treatment instrument.
